(19) United States
(12) Patent Application Publication
(10) Pub. No.: US 2024/0269676 A1
(43) Pub. Date: Aug. 15, 2024

(54) MICROFLUIDIC DEVICE, AND ASSOCIATED APPARATUS AND METHODS

(71) Applicant: IMPERIAL COLLEGE INNOVATIONS LIMITED, London (GB)

(72) Inventors: Sam H. AU, London (GB); Julian F. ASHBY, London (GB)

(21) Appl. No.: 18/565,871

(22) PCT Filed: Jun. 1, 2022
(86) PCT No.: PCT/GB2022/051399

§ 371 Date: Nov. 30, 2023

(30) Foreign Application Priority Data
Jun. 4, 2021 (GB) 2108066.8

(51) Int. Cl. B01L 3/00; G01N 33/483

(52) U.S. Cl. CPC B01L 3/502761; G01N 33/4833; B01L 2200/0652; B01L 2200/12; B01L 2400/0487

(57) ABSTRACT

The present disclosure relates to a microfluidic device for discriminating between second cells, as a function of binding strength between first cells and the second cells, the device comprising: a microfluidic channel, a base of the channel comprising a substrate; wherein the channel is configured to receive first cells, for adhesion of the first cells to the substrate; receive second cells, for adhesion of the second cells to the first cells; and receive a fluid, for fluid flow over the second cells; and wherein a transverse cross-section of the channel is configured for application of a substantially uniform shear stress to the second cells that are adhered to the first cells in at least a central region of the channel as the fluid flows along the channel, to detach at least some of the second cells from the first cells, and to cause the detached second cells to flow towards a downstream end of the channel.

### FIELD OF THE INVENTION

The invention relates to a microfluidic device, and associated apparatus and methods, for discriminating between cells based on avidity—in particular for discriminating between cells as a function of binding strength between the cells. Merely by way of example, the invention may be used to discriminate between T cells as a function of binding strength to tumour cells. Associated apparatus and methods are also provided.

It will be appreciated that the term ‘binding strength’ as used herein should be interpreted broadly to encompass cellular avidity and the overall strength of cell-to-cell interactions.

### BACKGROUND TO THE INVENTION

T cell receptor (TCR) T cell therapy is emerging as a promising mode of cancer immunotherapy. With this approach cytotoxic T cells can be engineered or selected based on TCR avidity (i.e. binding strength) to antigenic peptides or fragments thereof presented by the major histocompatibility complex (pMHC) present on tumour cells. Selected T cells are then expanded and re-injected to specifically target tumour cells with enhanced potency. Clinical trials of TCR T cell therapies have demonstrated efficacious anti-tumour responses for patients with metastatic melanoma, synovial sarcoma, and myeloma.

The binding strength between immune proteins and their targets are related to their ability to trigger an immune response. A major hurdle with TCR therapies is the selection of optimal TCRs among the exceptionally diverse human TCR repertoire, with billions of distinct TCR clonotypes. Existing technologies available to reliably detect rare T cell clones with optimal avidity are time-consuming, expensive and inefficient. Researchers have previously used surface plasmon resonance (SPR) to detect molecular kinetics and affinity of TCR-pMHC binding. However, affinity readouts disregard the interactions of multiple TCR-pMHC complexes and co-receptors. This results in poor predictions of T cell functionality. Progress in this field led to the development of pMHC multimer ortetramer staining that generates avidity readouts using flow cytometry. This technique provides high-throughput analysis of multiple TCR-pMHC interactions as well as their CD8 co-receptor. In particular, novel NTA-His tag-containing multimer technology (NTAmers) generates direct measurements of TCR-pMHC dissociation rates (k_{off}) that effectively evaluate the impact of avidity on antitumor responses for a wide range of antigen-specific T cell clones. On the other hand, multimer/tetramer staining commonly fails to detect functional T cells resulting in false negative readouts of bulk populations.

Moreover, all of the above technologies fail to account for potentially important cell-cell interactions such as integrin binding, gap junction proteins and other co-receptors that form the immunological synapse (IS). These additional interactions are known to contribute significantly to T cell binding, activation and binding. Therefore, the field requires novel strategies in order to evaluate the overall strength of cellular interactions between activated T cells and tumour cells, described here as cellular avidity.

Numerous studies have revealed that strong TCR-pMHC interactions typically result in superior anti-tumour responses. This is important for TCR T cell therapy that aims to enhance T cell reactivity against tumours, as the vast majority of reactive endogenous T cells express receptors with weak TCR-pMHC avidity due to mechanisms of central and peripheral tolerance that eliminate high avidity T cells.

In contrast, studies have revealed that T cells engineered with supraphysiological affinity/avidity lead to poor functionality and greater risk of cross-reactivity to self-antigens resulting in autoimmune diseases. Thus, there lies an optimal balance between improved anti-tumoral activity and autoimmunity that need to be carefully considered in order to achieve desired therapeutic outcomes.

Microfluidics enables the evaluation of immune responses in vitro with numerous advantages: (i) ability to analyse cell-cell interactions over time, (ii) real-time functional monitoring (e.g. activation and cytokine release) and (iii) precise control over the local microenvironment. Researchers have previously used droplet microfluidics to co-encapsulate tumour cells and TCR T cells into subnanolitre droplets using a microfluidic droplet generator. This technique identifies rare TCR T cell clones at the single-cell level under high-throughput by analysing GFP activation. However, this approach does not directly assess the strength of T cell-tumour cell interactions contributing to the cellular avidity. Furthermore, T cell activation readouts are slow and potentially impractical for examining primary T cells extracted from cancer patients.

It will be appreciated, therefore, that there is a need for better tools and techniques to more efficiently and accurately discriminate between cells based on avidity.

### SUMMARY OF THE INVENTION

Aspects of the present invention are set out in the appended independent claims, while details of certain embodiments are set out in the appended dependent claims.

According to a first aspect of the invention there is provided a microfluidic device as defined in Claim 1 of the appended claims.

Thus there is provided a microfluidic device for discriminating between second cells, as a function of binding strength between first cells and the second cells, the device comprising: a microfluidic channel, a base of the channel comprising a substrate; wherein the channel is configured to receive first cells, for adhesion of the first cells to the substrate; receive second cells, for adhesion of the second cells to the first cells; and receive a fluid, for fluid flow over the second cells; and wherein a transverse cross-section of the channel is configured for application of a substantially uniform shear stress to the second cells that are adhered to the first cells in at least a central region of the channel as the fluid flows along the channel, to detach at least some of the second cells from the first cells, and to cause the detached second cells to flow towards a downstream end of the channel.

By virtue of the transverse cross-section of the channel being configured as above, a relatively wide region of substantially uniform shear stress at the plane at which the first and second cells contact forms in the channel as fluid flows through the channel. Beneficially, this allows a large fraction of the second cells in the channel to be subjected to the same substantially uniform shear stress, thus enabling accurate discrimination between different sets of the second cells that detach from the first cells at different shear stresses. The detached second cells can be collected at the downstream end of the channel, which enables further analysis and/or propagation of the cells. Beneficially, therefore, the microfluidic device enables a set of the second cells, detached at substantially the same uniform shear stress, to be collected at the downstream end of the channel, allowing the avidity of those cells to be determined. Alternatively, or in addition, the second cells that remain adhered to the first cells in the channel following the application of said substantially uniform shear stress may be analysed.

Beneficially, the present work enables the avidity between the two sets of cells to be studied, in contrast to conventional techniques which do not take into account all of the interactions between the cells (for example, in techniques where cells may be bonded to acellular material).

Preferably, a width of the channel is between 10 and 50 times greater than a height of the channel, for example 10 times, 20 times or 30 times greater than the height of the channel. Beneficially, by configuring the width of the channel to be between 10 and 50 times greater than the height of the channel, a particularly wide region of substantially uniform shear stress is formed as the fluid flows through the channel.

Optionally, the channel may have a generally rectangular transverse cross-section.

Preferably, the microfluidic device further comprises a first flow path arranged to convey fluid from the central region of the channel into a collection outlet; a second flow path arranged to convey fluid from a first side region adjacent a first side wall of the channel into a waste outlet; and a third flow path arranged to convey fluid from a second side region adjacent a second side wall of the channel into a waste outlet. Beneficially, therefore, fluid from a central region of the channel (carrying the second cells that detached in that region) flows into the collection outlet, whereas fluid that flows near the side walls of the channel (and therefore flows in a region of non-uniform shear stress) flows into the waste outlet(s). This ensures that only cells that are subject to the substantially uniform shear stress pass into the collection outlet, thereby improving the accuracy of the determination of the avidity of the detached cells.

Preferably, the first flow path is coupled to the central region of the channel at the downstream end of the channel, the second flow path is coupled to the first side region of the channel at the downstream end of the channel, and the third flow path is coupled to the second side region of the channel at the downstream end of the channel.

Optionally, the transverse cross-section of the channel and the configuration of the first, second and third flow paths are such that the collected cells are subjected to less than 1% deviation in shear stress.

Optionally, the uniform shear stress is between 1.9 Pa and 19.2 Pa. Beneficially, these values of shear stress are low enough such that the first cells are prevented from becoming detached or killed, but high enough to allow for discrimination between cells of differing avidity.

Optionally, a fluid resistance of the first flow path relative to fluid resistances of the second and third flow paths is configured for flow of a predetermined fraction of the fluid into the waste outlet(s). In particular, the second and third flow paths may have greater fluid resistance than the first flow path.

The term ‘fluid resistance’ as used herein refers to the resistance to the flow of fluid along a flow path. The fluid resistance of a microfluidic flow path depends, for example, on its length and transverse cross-sectional area.

Thus, in some examples, the dimensions of the first flow path, the dimensions of the second flow path and the dimensions of the third flow path may be configured for flow of the predetermined fraction of the fluid into the first, second and third flow paths.

Optionally, the second and third flow paths are longer and/or have smaller transverse cross-sectional areas than the first flow path.

The second and third flow paths may be connected to a common waste outlet. Alternatively, the second and third flow paths may be connected to separate waste outlets.

In one particularly advantageous example, the device is configured such that fluid that flows within a predetermined distance from the side walls of the channel is diverted into the waste outlet(s), thereby disposing of the cells in such side regions that are subjected to a particularly non-uniform distribution of shear stress, close to the edges of the channel. The predetermined distance may be 10% of the width of the channel. The predetermined distance may be 0.1 mm.

Optionally, the relative fluid resistances of the first, second and third flow paths are configured such that 80 percent of the fluid that flows along the channel flows into the collection outlet, and the remaining 20 percent of the fluid that flows along the channel flows into the waste outlet.

In one example, the first cells are T cells, for example human T cells, and the second cells are tumor cells, for example human tumor cells. Alternatively, the first cells may be tumor cells, for example human tumor cells, and the second cells may be T cells, for example human T cells.

Preferably, the height of the channel is less than 0.5 mm, for example 0.1 mm.

Beneficially, this reduces the time required for the first cells to adhere to the substrate, and also reduces the time (and narrows the temporal distribution—the distribution of time required for each second cell to attach to a corresponding first cell) required for the second cells to adhere to the first cells.

Optionally, the substrate may be modified with a plasma treatment or other non-protein based treatment.

Optionally, the substrate may be a plastic substrate.

Optionally, the substrate may be a glass substrate. Optionally, the glass substrate may be coated with poly-L-lysine or fibronectin, which are particularly beneficial since they adhere strongly to glass and promote the adhesion of first cells to substrates.

Optionally, the fluid may comprise at least one of a biological fluid, balanced salt solution, basal media, complex media, a mineral or other cell-compatible oil.

Optionally, the fluid may comprise a shear thinning fluid (for example, blood) or a shear thickening fluid.

Preferably, the first cells form a monolayer on the substrate, providing a large surface area with which the second cells may adhere to. Preferably, the length of the channel is greater than 10 mm, for example 50 mm, providing a large region in which the cell-substrate and cell-cell adhesion may occur.

According to a second aspect of the invention there is provided apparatus comprising the microfluidic device of the first aspect.

Optionally, the apparatus further comprises a controller for controlling a flow rate of the fluid into the channel to control the substantially uniform shear stress.

Beneficially, the provision of the controller allows the flow rate of fluid into the channel, and therefore the shear stress applied to the second cells, to be precisely controlled. In a particularly advantageous embodiment, the apparatus comprises a syringe pump (for example, a programmable syringe pump) for controlling the flow rate of the fluid into the channel to control the substantially uniform shear stress applied to the second cells.

Optionally, the apparatus further comprises a sorting stage towards the downstream end of the channel for sorting groups of the second cells based on the applied shear stress at which the second cells detached from the first cells. Beneficially, this enables groups of the second cells that detached at a predetermined shear stress to be separated for further downstream analysis. However, it will be appreciated that the second cells that detach from the first cells need not necessarily be collected and analysed, since alternatively the second cells that remain attached to the first cells in the channel could instead by analysed (for example, but imaging the cells in the channel). Optionally, the apparatus may further comprise a downstream analysis stage for analysing the sorted groups of detached second cells. Optionally, the apparatus may further comprise a propagation stage for propagating the detached second cells.

Preferably, the controller is configured to sequentially change the flow rate of the fluid into the channel between a plurality of predetermined flow rates. This feature may be particularly beneficial in combination with the sorting stage, as this provides a particularly efficient way in which to collect and separate (i.e. discriminate between) groups of the second cells having different avidities.

Optionally, the sorting stage is configured to sort the second cells into groups of the second cells that are detached from the first cells at each of the corresponding predetermined flow rates.

Preferably, the apparatus further comprises an imaging device configured for imaging the first and/or second cells in the channel. Beneficially, this enables the second cells that detach from the first cells at a predetermined shear stress to be identified, and/or the cells that remain attached to the first cells in the channel after application of the predetermined shear stress to be identified.

According to a third aspect of the invention there is provided method as defined in Claim 34 of the appended claims.

Thus there is provided a method for discriminating between second cells, as a function of binding strength between first cells and the second cells, the method comprising, in a microfluidic channel, a base of the channel comprising a substrate: receiving first cells, for adhesion of the first cells to the substrate, receiving second cells, for adhesion of the second cells to the first cells; and receiving a fluid, for fluid flow over the cells; wherein a transverse cross-section of the channel is configured for application of a substantially uniform shear stress to the second cells that are adhered to the first cells in at least a central region of the channel as the fluid flows along the channel, thereby detaching at least some of the second cells from the first cells, and causing the detached second cells to flow towards a downstream end of the channel.

Preferably, the second cells are allowed to adhere to first cells for a period of between 5 and 10 minutes, for example 10 minutes, under static conditions (i.e. without a substantial applied shear stress). Beneficially, this period ensures that sufficient adhesion occurs between the first cells and the second cells, without comprising cell viability or forming overly-strong bonds.

Optionally, the method may further comprise sorting groups of the second cells based on the applied shear stress at which the second cells detached from the first cells. Optionally, the method may further comprise analysing the sorted groups of detached second cells. The analysing may include determining an avidity between at least one of the second cells and the first cells based on the applied shear stress at which the at least one second cell detached.

Embodiments of the invention may include a collection system for sorting cells and may include a programmable syringe pump that induces uniform shear flow over cells in the device. This enables one to discriminate and collect cells based on their binding strength to the substrate or other cells. In one example, collected T cells are separated based on their cell avidity to tumour cells adhered to the device. This provides a means of assessing T cell avidity and collecting these T cells for further downstream functional analysis including cytotoxicity, proliferation and cytokine/chemokine secretion. This step is beneficial for researchers and clinicians to further quantity T cell functionality and provides the capability of retrieving rare high avidity patient derived T cells out of heterogeneous populations.

It will be appreciated, therefore, that present disclosure provides improved apparatus and methods for probing cellular avidity. In contrast, currently deployed methods for selecting TCR clonotypes based on the avidity of TCR and pMHC interactions are acellular and therefore incapable of probing important physiological cues for T-cell activation including a range of co-receptors and cell-cell interactions such as integrins and gap junction proteins. The superior optical properties of microfluidic devices and the ability to operate under physiological conditions allows the automated real-time monitoring of tens of thousands of live T cell and tumour cell interactions per device. The apparatus and methods could also be used for other applications such as validation of selected TCR clonotypes or evaluation of combinatorial immunotherapy with chemotherapies, drug-loaded lipid nanoparticles, nanogels or others. Moreover, current strategies to find cells with optimal avidity to neoantigens is even more laborious than self-antigens, as every patient expresses different neoantigens. This platform could potentially accelerate the identification of suitable patient-specific neoantigen T cell candidates.

### BRIEF DESCRIPTION OF THE DRAWINGS

Embodiments of the invention will now be described by way of example only with reference to the attached figures in which:

FIG. 1 schematically illustrates an overview of a microfluidic device;

FIG. 2 schematically illustrates a more detailed view of an outlet portion of the microfluidic device of FIG. 1;

FIG. 3 schematically illustrates a fluid dynamic simulation of shear stress distribution across the width of a channel of the microfluidic device as a result of fluid flow along the channel;

FIG. 4 shows a graph of shear stress against position across the width of the channel;

FIG. 5 schematically illustrates cells inside the channel of the microfluidic device;

FIG. 6 schematically illustrates an interaction between two cells;

FIGS. 7a to 7d schematically illustrate an effect of increasing fluid flow rate through the microfluidic device, showing the progressive detachment of cells having varying functional avidities with increasing flow rate and thus increasing shear stress:

FIG. 8 schematically illustrates a method using the microfluidic device;

FIG. 9 shows representative images of Me290, B16F10 and NA8 cell attachment under 0-19 Pa shear stresses:

FIG. 10 shows a graph of melanoma cell adhesion Me290, NA8 and B16F10 against shear stress;

FIG. 11 shows a graph of melanoma cell viability against shear stress;

FIG. 12 shows representative calcium flux time-lapse images of DMP T cell activation on Me290 melanoma monolayer;

FIG. 13 shows a graph of SupT1 T cell activation against melanoma cell type;

FIG. 14 shows representative images of SupT1 T cell detachment from Me290 melanoma cells;

FIG. 15 shows representative images of SupT1 T cell detachment from NA8 melanoma cells;

FIG. 16 shows a graph illustrating quantification of SupT1 T cells with varying avidities (DMβ with high avidity, WT with intermediate avidity and NT with weak avidity) to attached Me290 cells under shear-induced flow;

FIG. 17 shows a graph illustrating quantification of SupT1 T cell attachment to NA8 melanoma cells and monolayer-free devices;

FIG. 18 shows a graph illustrating quantification of SupT1 T cells collected from the product outlet of the device under increasing shear stress;

FIG. 19 shows a graph illustrating percentage of each SupT1 T cell variant collected;

FIG. 20 schematically illustrates mouse T-cell isolation, expansion and seeding;

FIG. 21 shows a graph illustrating quantification of Pmel (high avidity) and B6 T cells (weak avidity) attachment to B16F10 melanoma cells under increasing shear stress;

FIG. 22 shows a graph illustrating quantification of primary mouse T cell collection from the product outlet under increasing shear stress;

FIG. 23 shows a further graph of primary mouse T cell collection against shear stress;

FIG. 24a shows a graph illustrating cytotoxicity of primary T cell fractions collected from the device outlet based on their cellular avidity to B16F10 melanoma cells;

FIG. 24b shows quantification of T cell activation by calcium flux indicator from sorted T cell fractions and unsorted T cells on B16F10 melanoma monolayers;

FIG. 24c shows quantification of persistence of cellular avidity after second device challenge;

FIG. 25a shows quantification of the mean fluorescent intensity ratio of Connexin 43 (Cx43) accumulation at the immunological synapse normalised to the opposite side of cells after high, low and no shear-induced flow conditions, with 25 cell conjugates analyzed per condition; and

FIG. 25b shows a graph of IFN-γ secretion of T cell-tumour cell co-culture media collected at the device outlet.

In the figures, like elements are indicated by like reference numerals throughout.

### DETAILED DESCRIPTION OF PREFERRED EMBODIMENTS

The present embodiments represent the best ways known to the Applicant of putting the invention into practice. However, they are not the only ways in which this can be achieved.

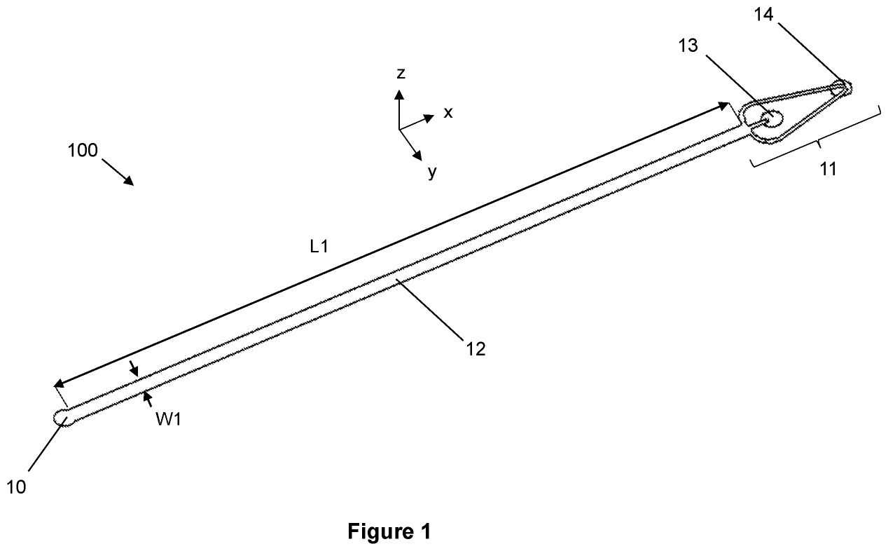

With reference initially to FIG. 1, the present disclosure provides a microfluidic device 100 for discriminating between second cells, as a function of binding strength between first cells and the second cells.

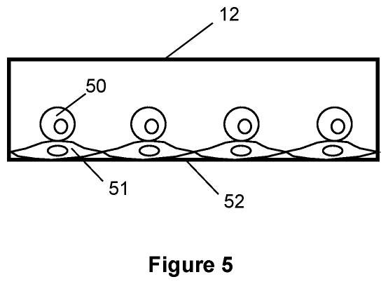

More particularly, the device 100 comprises a microfluidic channel 12 that extends between a fluid inlet 10 and an outlet portion 11. Turning briefly to FIG. 5, the device is configured to receive first cells 51 for adhesion to a substrate 52, and to receive second cells 50 for adhesion of the second cells 50 to the first cells 51. As illustrated in FIG. 5, the substrate 52 may be provided on the base (i.e. lower surface/wall) of the channel. The device 100 is further configured to receive a fluid for fluid flow over the cells. For example, the device may receive the fluid via the inlet 10 from an inlet reservoir.

In this example, the channel 12 is 1 mm wide (in the y direction, as indicated by W1), has a height 0.1 mm (in the z direction), and has a length of 50 mm (in the x direction, as indicated by L1). Beneficially, the transverse cross-section of the channel 12 (i.e. the cross-section in the Y-Z plane) creates a large region of substantially uniform shear stress in a central region of the channel 12, as fluid flows along the channel 12 towards the downstream end. In this example, the channel 12 has a generally rectangular transverse cross-section. However, it will be appreciated that this need not necessarily be the case, and any other suitable shape of transverse cross-section may be used.

Whilst in the example shown in FIG. 1 the channel 12 is a straight channel extending between the inlet 10 and the outlet portion 11, the channel 12 need not necessarily be straight. For example, a serpentine or curved channel may be provided between the inlet 10 and the outlet portion 11.

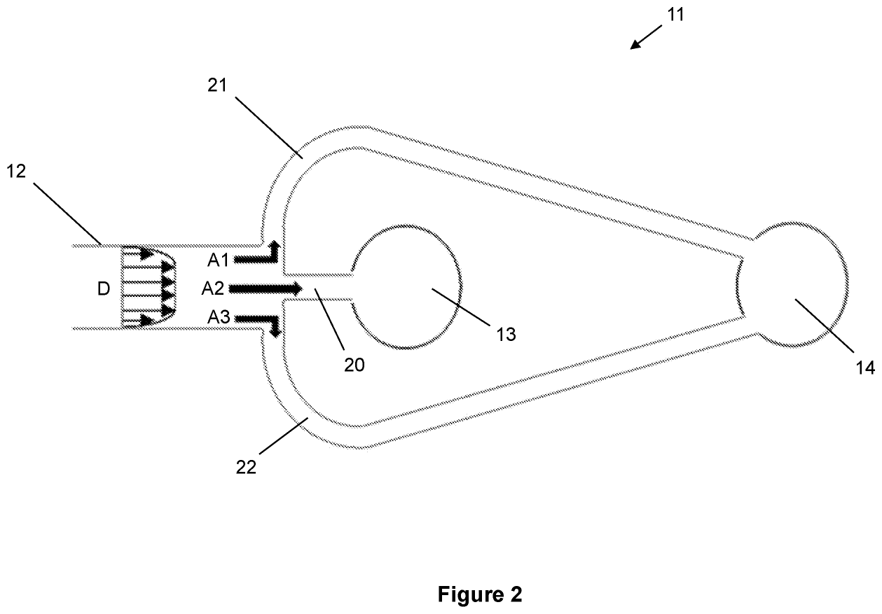

The channel 12 is configured to receive a fluid via the fluid inlet 10, for flow along the channel 12 to the outlet portion 11 at the downstream end of the channel 12. The outlet portion 11 is illustrated in more detail in FIG. 2. As illustrated in the Figure, a fluid flows (as indicated by the group of arrows D) through the channel 12 towards the outlet portion 11 located at the downstream end of the channel 12. As illustrated by the arrows D, the fluid has a parabolic flow profile as it flows through the channel 12 towards the downstream end. The channel 12 is coupled to first 20, second 21 and third 22 flow paths. The first flow path 20 is arranged to convey fluid from a central region of the channel 12 (as indicated by arrow A2) into a collection outlet 13. The second flow path 21 is arranged to convey fluid from a first side region adjacent a first side wall of the channel 12 (as illustrated by arrow A1) into a waste outlet 14. The third flow path 22 is similarly arranged to convey fluid from a second side region adjacent a second side wall of the channel 12 (as illustrated by arrow A3) into the waste outlet 14. Beneficially, fluid that flows in the regions adjacent to the side walls of the channel 12 (and therefore does not apply a uniform shear stress to cells in the channel, as described in more detail below) is diverted away from flowing into the collection outlet 13.

In the illustrated example, the second 21 and third 22 flow paths are configured to convey fluid from the channel 12 to a common waste outlet 14. Alternatively, the second 21 and third 23 flow paths may be configured to convey fluid to two separate waste outlets.

Beneficially, the relative fluid resistances of the first 20, second 21 and third 22 flow paths are configured for a flow of a predetermined fraction of the fluid exiting the downstream end of the channel 12 into the collection outlet 13 (and a corresponding predetermined fraction of the fluid into the waste outlet 14). More specifically, the fluid resistance of the first flow path 20 between the channel 12 and the collection outlet 13 relative to the fluid resistance of the second 21 and third 22 flow paths between the channel 12 and the waste outlet 14 is configured for a flow of a predetermined fraction of the fluid exiting the downstream end of the channel 12 into the collection outlet 13 (and a corresponding predetermined fraction of the fluid into the waste outlet 14).

It will be appreciated that the fluid resistance of each flow path will depend on the dimensions of that flow path. Therefore, the dimensions of the first flow path 20, second flow path 21 and third flow path 22 are configured for flow of the predetermined fraction of fluid that flows into the collection outlet 13 and the predetermined fraction of fluid that flows into the waste outlet 14. For example, the relative lengths and transverse cross-sectional areas may be configured for flow of the predetermined fraction of fluid into the collection outlet 13 and the predetermined fraction of fluid into the waste outlet 14. In the illustrated example, the second and third flow paths are longer than the first flow path. Alternatively, or additionally, the second and third flow paths may have smaller transverse cross-sectional areas than the first flow path.

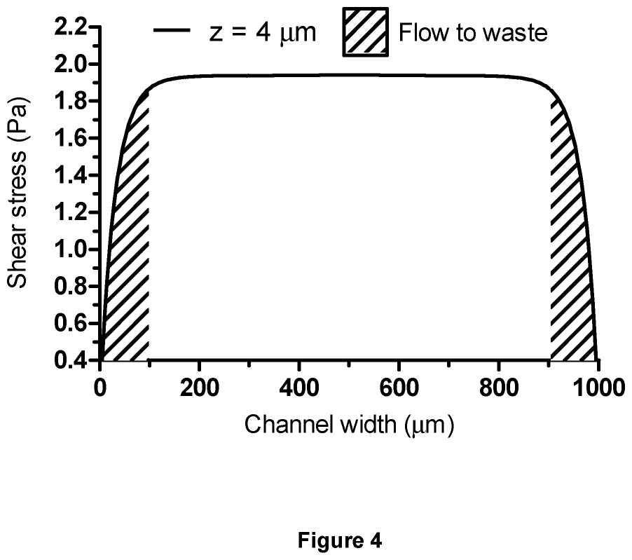

Advantageously, in the illustrated example, the first 20, second 21 and third 22 flow paths are configured such that fluid that flows within 10% of the width of the channel (in this example, 0.1 mm) from the side walls of the channel flows into the waste outlet 14, and the remaining fluid flows into the collection outlet 13. In other words, fluid that flows within 0.1 mm from the side walls of the channel is prevented from flowing into the collection outlet 13. As discussed below in relation to FIG. 4, it is beneficial to discard cells located in such side regions near the side walls of the channel 12, since these cells are subjected to a particularly non-uniform distribution of shear stress, and would broaden the distribution of shear stress applied to the cells that flow into the collection outlet 13, and thus lead to a less accurate determination of avidity.

In the illustrated example, the relative fluid resistances of the first 20, second 21 and third 22 flow paths are configured such that 80 percent of the fluid that flows along the channel 12 flows into the collection outlet 13, and the remaining 20 percent of the fluid that flows along the channel 12 flows into the waste outlet 14 (i.e. 10 percent from each side of the channel 12).

Whilst in the illustrated example the first 20, second 21 and third 22 flow paths are coupled to the far end of the channel 12 (i.e. the far downstream end of the channel), it will be appreciated that this need not necessarily be the case. For example, the second 21 and third 22 flow paths may be coupled to the channel 12 upstream of the first channel 12, provided that there is no detachment of cells that occurs in the channel 12 between the location at which the fluid enters the second 21 and third 22 flow paths and the location at which the fluid enters the first flow path 20 (to prevent cells detaching under non-uniform shear stress between the flow paths to the waste outlet 14 and the flow paths to the product outlet 13).

A plurality of the channels 12 may be provided in order to provide a larger region for the cell-substrate and cell-cell interactions to occur. Each of the plurality of channels 12 may be connected a common fluid inlet 10. Fluid flowing out of the collection outlet of each of the plurality of channels 12 may flow into a common collection reservoir.

The microfluidic device 100 is compatible with a broad range of fluid types. Fluid selection depends on the objective of the application; however, for cell-based applications this typically requires a balanced salt solution, with physiological pH and osmotic pressure to maintain cell viability during live-cell experiments. Non-limiting examples of compatible fluid types include:
Biological fluids (e.g. plasma, serum, and lymph)
Balanced salt solutions (e.g. PBS, DPBS, HBSS, EBSS)
Basal media (e.g. DMEM)
Complex media (e.g. RPMI-1640, IMDM)
Other types of Newtonian fluids (e.g. water)
Mineral and other cell-compatible oils
Shear thinning fluids (e.g. blood)
Shear thickening fluids

As described in more detail below, e.g. with reference to FIG. 5, the channel 12 comprises a substrate 52 for adhering to a set of first cells 51. The first cells 51 may be received via the inlet 10, for adhesion of the first cells 51 to the substrate 52. The substrate 52 may extend along substantially the entire length of the channel 12, or may extend along only a portion of the length of the channel 12. In this example, the substrate 52 is provided along the lower surface of the channel 12 (i.e. the base of the channel). A set of second cells 50 may then be received via the inlet 10, for adhesion to the first cells 52 that are bonded to the substrate 52.

In this example, the substrate 52 is a glass substrate, which may be coated, for example, with poly-L-lysine or fibronectin. In the present example, a polydimethylsiloxane (PDMS) part is plasma bonded to a glass substrate to form the microfluidic device 100. The glass substrate functions as a solid surface to attach coatings followed by adhering tumour cells (first cells 51) for cell avidity studies. However, there are many substrate materials that could be used depending on the application including:
Glass (e.g. microscope glass slides and coverslips)
Plastics: e.g. polydimethylsiloxane (PDMS), polystyrene (PS), cyclic olefin copolymer (COC), polyethylene terephthalate (PET), polycarbonate (PC), polytetrafluoroethylene (PTFE), thermamox (TPX), polyvinylchloride (PVC), polyacrylamide, and polymethyl methacrylate (PMMA)
Silicon
Quartz
Palladium
Hydrogels (collagen, gelatin, Matrigel™, PEGDA, GelMA, agarose, agar, others)

The device substrate can be coated with a broad range of adhesion proteins or attachment factors, depending on the cell type and substrate material used for a specific application. For example, one or more of the following coatings may be applied to the substrate:
Poly-L-lysine
Poly-D-lysine
Poly-L-ornithine
Fibronectin
Collagen (types 1/II/III/IV)
Laminin
Vitronectin
Gelatin
Cadherin
Selectin
Engineering proteins or peptides with RGD-binding domains
Proteoglycans

The poly-L-lysine and fibronectin coatings are particularly beneficial when a glass substrate is used, since they adhere strongly to the glass substrate. Substrates may be subjected to other pre-treatment/modification steps to improve the attachment of coatings to the surface and/or promote adhesion of first cells directly to the substrates. These pre-treatment/modification steps may include:
Plasma treatment (typically oxygen, but can be done with other gases and air)
Silanization (3-aminopropyl)triethoxysilane (APTES)
Polydopamine (PDA)
Polyethylene glycol (PEG)
Surface roughening or micropatterns to promote adhesion (for instance via the formation of pillars, microstructures, nanostructures, oxygen plasma treated and/or ion-beam etching surface roughening)

Some of these pre-treatment/modification steps may reduce or remove the need for protein-based coatings to be applied to the substrate.

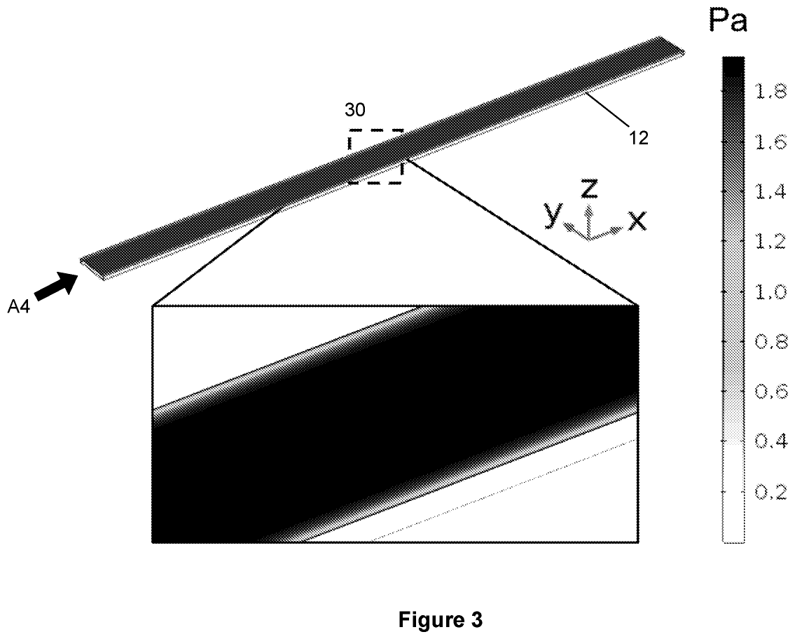

FIG. 3 shows a fluid dynamic simulation shear stress contour plot at 200 μL/min flow rate in middle of device 100 at 4 μm above the surface (to represent the height of a T cell-tumour cell interaction). As described in more detail below, as fluid flows along the channel 12, the transverse cross-section of the channel 12 results in the application of a substantially uniform shear stress to the second cells that are adhered to the first cells in at least a central region of the channel 12. As illustrated in the figure, a flow of fluid enters the channel 12 at the upstream end via the inlet 10, as illustrated by arrow A4. A magnified view of the region of the channel indicated by the dashed box 30 is shown. The scale indicates the shear stress that is applied to the second cells at the typical height of adhesion to first cells (a height of 4 μm above the base of the channel 12) as the fluid flows along the channel 12 at a particular flow rate. As can be seen in the figure, there is a large region of substantially uniform shear stress in a generally central region of the channel 12. The region of substantially uniform shear stress extends across a major fraction of the width of the channel 12. Beneficially, as described in more detail below, the large region of substantially uniform shear stress allows a large number of the second cells 50 to be detached from the first cells 51 at a known constant shear stress, for subsequent downstream analysis.

It will be appreciated that for a given fluid and a specific height above the base of the channel 12, the shear stress can be derived from a given flow rate of fluid along the channel 12.

FIG. 4 shows a graph of the shear stress against the position across the width of the channel 12 (i.e. against the y-position indicated in FIG. 1), corresponding to the fluid dynamic simulation of FIG. 3, at a height (z-position) of 4 μm above the base of the channel 12 (the magnitude of the shear stress increases away from the base of the channel, i.e. for larger values of z). As illustrated in the figure, there is a large central region of substantially uniform shear stress. It will be appreciated that the term ‘substantially uniform shear stress’ is used throughout this specification to refer to a shear stress that is substantially uniform, but not necessarily exactly constant. In other words, the shear stress has a low percentage deviation, for example less than 1%. Either side of the central region of substantially uniform shear stress there are two regions (each of 100 μm in width) corresponding to a lower and non-uniform shear stress due to the interaction between the fluid and the side walls of the channel 12 (indicated by the shaded regions of the figure). Beneficially, the configuration of the first 20, second 21 and third 22 fluid paths at the downstream end of the channel 12 is arranged to divert the fluid that flows along these side regions of the channel 12 into the waste outlet 14, whereas the flow along the central region of the channel (corresponding to the region of uniform shear stress) is diverted into the collection outlet 13. In use, when the applied shear stress reaches a sufficiently high value, the shear stress will cause at least some of the second cells 50 to detach from the first cells 51. The second cells will then flow, with the fluid, towards the downstream end of the device and into either the collection outlet 13 or the waste outlet 14, depending on the y-position across the width of the channel. It will be appreciated, therefore, that the configuration of the first 20, second 21 and third 22 fluid paths allows the collection of second cells that have been subjected to a substantially uniform shear stress at the collection outlet 13, whereas the second cells from the peripheral regions of the channel 12 that have been subjected to a non-uniform shear stress are diverted to the waste outlet 14.

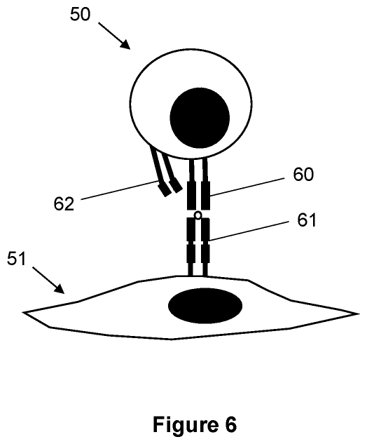

FIG. 5 shows a schematic illustration of a region inside the channel 12. As shown in the figure, a monolayer of the first cells 51 is attached to a substrate 52 on the base of the channel. As shown in the figure, the first cells form a monolayer of cells (i.e. a layer of cells that is one cell thick) on the surface of the substrate. The second cells 50 are attached to the first cells 51. In this example, the first cells 51 are tumour cells and the second cells 50 are T cells. Examples of experiments using specific types of tumour cells and T cells are provided in more detail below (a bonding interaction between a T cell 50 and a tumour cell 51 is illustrated in FIG. 6, and is also described in more detail below). The fluid that flows along the channel 12 may comprise at least one of a biological fluid, balanced salt solution, basal media, complex media, a mineral or other cell-compatible oil, or any other suitable fluids. It will be appreciated that the choice of fluid may depend on the type of first cell that is adhered (bonded) to the substrate and the type of second cell that is adhered to the first cells. The fluid may comprise a shear thinning fluid (for example, blood) or a shear thickening fluid. It will be appreciated, therefore, that the choice of fluid may also depending on the desired flow characteristics through the channel 12.

The ratio of the width of the channel 12 (in the y-direction of FIG. 1) to the height of the channel (in the z-direction of FIG. 1) is an important factor in achieving a large region of substantially uniform shear stress. As described above, in this example the channel 12 is 1 mm wide and the height of the channel is 0.1 mm, resulting in a 10:1 ratio between the width and the height of the channel. Beneficially, the relatively large width of the channel compared to the height of the channel results in a large region of substantially uniform shear stress as the fluid flows along the channel. Nevertheless, the channel 12 is not limited to using a ratio of 10:1 between the width and height of the channel (although this ratio may be particularly beneficial for providing a large region of substantially uniform shear stress). For example, a ratio of 20:1 or 30:1 could be used. However, the absolute values of the width of the channel and the height of the channel should also be considered. For example, if the channel 12 is formed of a flexible material (e.g. a plastic material), then a large width of the channel 12 may result in ‘bowing’ at the centre of the channel. Moreover, if the height of the channel is particularly large, then there will be a particularly large region between the monolayer of first cells 51 on the substrate 52 at the base of the channel 12 and the upper wall (ceiling) of the channel. As a result, when the first cells 51 are introduced into the channel 12 to bond to the substrate 52, a longer interaction time may be required to form the monolayer. Similarly, when the second cells 50 are introduced into the channel 12 to bond to the first cells 51, a longer interaction time will be required for the second cells 50 to bond to the first cells 51 and a greater deviation in the time (i.e. broader distribution in the time taken for each second cell 50 to bond to a corresponding first cell 51) required to initiate contact between the first and second cells may result. Since the binding strength between the first 51 and second 50 cells may depend on the duration for which the second cells 50 are bonded to the first cells 51, it will be appreciated that a broader distribution in the time taken for each second cell 50 to bond to a corresponding first cell 51 may negatively affect subsequent downstream analysis of the cellular avidity.

In the example shown in FIG. 1, the length of the channel is 50 mm. Beneficially, a longer channel 12 allows a large region of substrate 52 to be provided, which provides a larger area for the first cells to bond to, and a larger area for the second cells 50 to bond to the first cells 51.

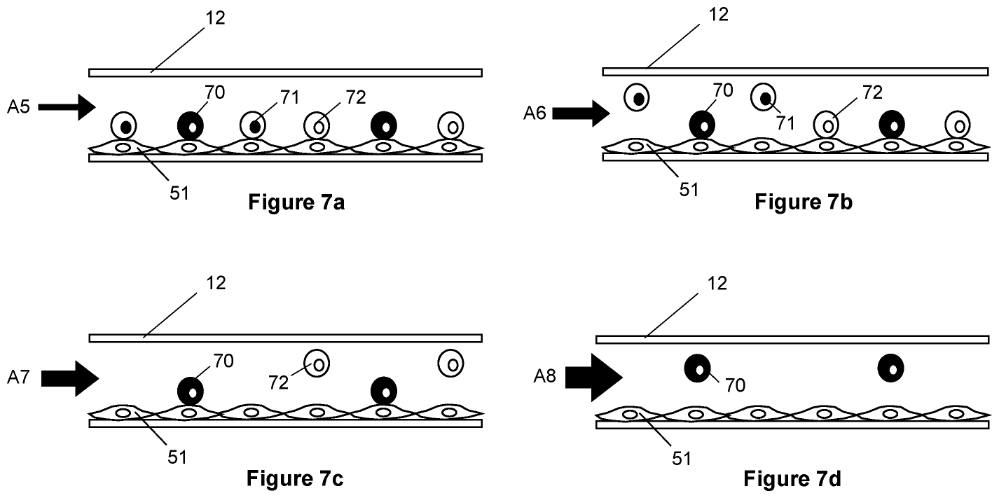

FIG. 7 shows a schematic illustration of second cells progressively detaching from the monolayer of first cells 51 at different applied shear stresses. In FIG. 7a, first 70, second 71 and third 72 types of T cell (i.e. three types of the second cells 50) are bonded to the monolayer of tumour cells 51 (which are bonded to the substrate 52 on the base of the channel 12).

In FIG. 7a, fluid flows along the channel 12, towards the downstream end, at a first flow rate indicated by arrow A5. The first flow rate is sufficiently low such that the shear stress applied to the second cells does not cause the second cells to detach from the first cells.

In FIG. 7b, fluid flows along the channel 12 at a second flow rate, greater than the first flow rate, as indicated by arrow A6. The increased flow rate increases the shear stress applied to the T cells, and results in the second type of T cell 71 becoming detached from the first cells. It will be appreciated, therefore, that the second type of T cell 71 has a relatively low avidity to the first cells 51 compared to that of the first 70 or third 72 types of T cell. The detached cells will flow, with the fluid, towards the downstream end of the channel 12, where they pass into either the collection outlet 13 (for flow from the central region of the channel) or the waste outlet 14 (for flow near the channel side walls). At the second flow rate, the first 70 and third 72 types of T cell remain adhered to the monolayer of tumour cells 51.

In FIG. 7c, fluid flows along the channel 12 at a third flow rate, greater than the second flow rate, as indicated by arrow A7. The increased flow rate further increases the shear stress applied to the T cells, and results in the third type of T cell 72 becoming detached from the first cells. The detached cells will flow, with the fluid, towards the downstream end of the channel 12, where they pass into either the collection outlet 13 (for flow from the central region of the channel) or the waste outlet 14 (for flow near the channel side walls). At the third flow rate, the first 70 type of T cell remains adhered to the monolayer of tumour cells 51. It will be appreciated, therefore, that the first 70 type of T cell in this example has a relatively high avidity compared to that of the second 71 or third 72 types of T cell.

In FIG. 7d, fluid flows along the channel 12 at a fourth flow rate, greater than the third flow rate, as indicated by arrow A8. The increased flow rate further increases the shear stress applied to the T cells, and results in the first type of T cell 70 becoming detached from the first cells. The detached cells will flow, with the fluid, towards the downstream end of the channel 12, where they pass into either the collection outlet 13 (for flow from the central region of the channel) or the waste outlet 14 (for flow near the channel side walls). At the fourth flow rate, all of the T cells have detached from the monolayer of tumour cells.

As described in more detail below, the microfluidic device 100 may be coupled to a device for causing the fluid to flow along the channel 12 at a plurality of predetermined flow rates. For example, the channel 12 may be connected to a programmable syringe pump at the inlet 10 for causing fluid to flow down the channel 12 at a plurality of predetermined flow rates. Alternatively, the channel 12 may be coupled to a different type of device for controlling the flow rate of fluid through the channel 12, such as a pressure controller. Alternatively, other methods may be used to control the flow of fluid along the channel, such as the provision of a material having a predetermined fluid resistance (e.g. a material having an appropriate hydrostatic head) at the inlet 10. In a particularly advantageous example, the device may be configured (e.g. programmed) to sequentially increase the flow rate (resulting in a corresponding sequential increase in the shear stress applied to the second cells) between a set of predetermined flow rates. A sorting stage (which may also be referred to as ‘sorting apparatus’) may be configured to sort the second cells 50 that flow through the collection outlet 13 based on the applied shear stress, in order to discriminate between groups of the second cells 50 that detached from the first cells 51 at different applied shear stresses. This beneficially allows improved downstream analysis of the detached second cells 50.

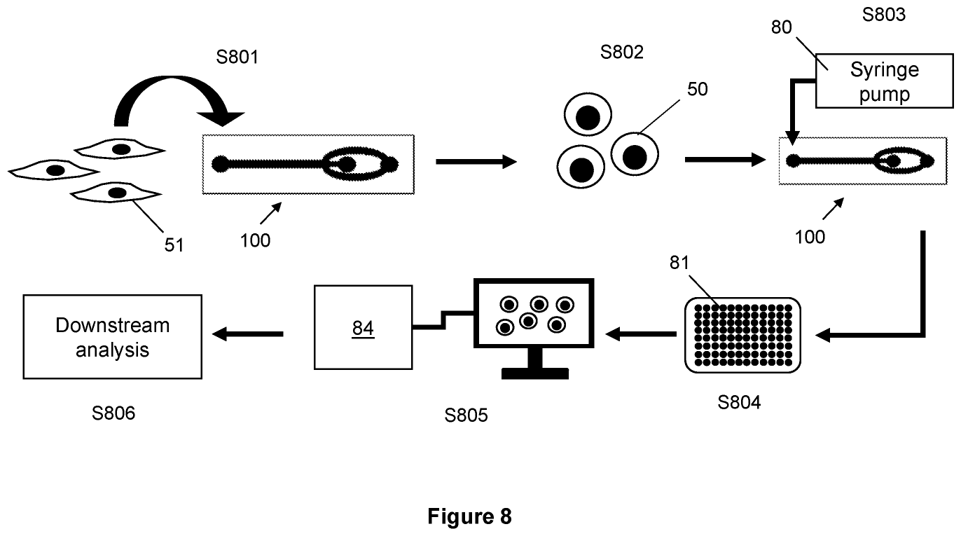

FIG. 8 shows a schematic flow diagram of a method of using apparatus comprising the microfluidic device 100. Aspects of the method will be described in more detail in the ‘further details and experimental validation’ section below.

In step S801, the first cells 51 are attached to the substrate 52 on the base of the channel 12. The first cells 51 may be introduced into the channel 12 by passing through the inlet 10. The first cells 51 are then left to bond to the substrate 52 under static conditions (i.e. without applied shear stress caused by fluid flowing through the channel 12). As discussed above, the time required for a sufficient number of the first cells 51 to bond to the substrate 52 (the cell-substrate bonding time) will depend on the height of the channel (the length of the channel in the z-direction of FIG. 1). For the channel height of 0.1 mm of the present example, a cell-substrate bonding time of between 30 and 60 minutes (for example, 45 minutes) may be used. This helps to ensure that the first cells 51 remain strongly bound to the substrate 52 under the applied shear stresses. However, it will be appreciated that the required cell-substrate bonding time will depend on the particular type of first cells 51 and the particular type of substrate 52 in use.

In step S802, the second cells 50 are introduced into the channel to bond to the first cells 51. The second cells 50 may be introduced into the channel 12 by flowing through the inlet 10. The second cells 50 are then left to bond to the first cells 51 under static conditions (i.e. without applied shear stress caused by fluid flowing through the channel 12). The time required for a sufficient number of the second cells 50 to bond to the first cells 51 (the cell-cell bonding time) will depend on the height of the channel (the length of the channel in the z-direction of FIG. 1). For the channel height of 0.1 mm of the present example, a cell-cell bonding time of between 5 and 15 minutes (for example, 10 minutes) may be used. This cell-cell bonding time was optimised to allow the cell-cell interactions to occur, whilst not being too long so as to compromise the cell viability, or form too-strong of an interaction for the immunological synapse. However, it will be appreciated that the range of suitable cell-cell bonding times will depend on the particular type of first cells 51 and the particular type of second cells 50 in use.

In step S803, a programmable syringe pump 80 is used to cause fluid to flow along the channel 12, over the cells inside the channel 12, towards the downstream end. To this end, a controller may be provided for controlling the flow rate of the fluid into the channel 12 to control the substantially uniform shear stress applied to the second cells 50. The flow of fluid causes a shear stress to be applied to the second cells 50, which may cause some of the second cells 50 to detach from the first cells 51 and flow with the fluid towards the downstream end. It will be appreciated that the shear stress required to detach the first cells 51 from the second cells 50 depends on the binding strength between the first cells 51 and the second cells 50. It will also be appreciated, therefore, that by controlling the applied shear stress and determining which of the second cells 50 detach from the first cells 51 (or alternatively, which of the second cells 50 remain attached to the first cells 51), the device 100 is able to discriminate between the first cells as a function of binding strength between the first cells 51 and the second cells 50. As described above with reference to FIGS. 7a to 7d, a plurality of discrete flow rates may be sequentially applied, to apply a series of stepwise increases in the shear stress applied to the second cells 50.

In step S804, the second cells 50 that flow into the collection outlet 13 are collected and sorted. For example, with reference to FIGS. 7a to 7d, the second cells 50 that detach at the four different flow rates (indicated by arrows A5 to A8) are separated. In this example, the collection outlet 13 is initially coupled to a first set of collection wells 81 (or a first collection reservoir), for example a standard 96-cell culture well plate, for flow of fluid (and the corresponding second cells 50 detached at the first applied shear stress) from the collection outlet 13 to the first set of collection wells 81. When the applied shear stress is increased using the pump 80, the collection outlet is instead coupled to a second set of collection wells 81 (or a second collection reservoir), for flow of fluid (and the corresponding detached second cells 50) from the collection outlet 13 to the second set of collection wells 81. It will be appreciated, therefore, that by controlling which set of collection wells 81 the collection outlet 13 is coupled to, the second cells 50 that detach from the first cells 51 at different applied shear stresses may be separated, and therefore that the apparatus is able to discriminate between the first cells as a function of binding strength between the first cells and the second cells. Preferably, the apparatus is configured to automatically couple the collection outlet 13 to different sets of collection wells according to the applied shear stress. Alternatively, the process may be performed manually by a user. The collection wells may be transferred to an incubator, where the T cells may be left to proliferate for further downstream analysis.

Beneficially, the apparatus may be used to perform downstream analysis of the sorted second cells. For example, an imaging device may be used to determine the type of second cells that detached at a specific shear stress. In this example, the apparatus further comprises an imaging device 84 (including, for example, a microscope) for imaging the collected detached second cells 50. Alternatively, an imaging device 84 (including, for example, a microscope) may be provided for imaging the cells inside the channel 12, in order to study the second cells 50 that remain adhered to the first cells 51 inside the channel 12. The imaging device 84 may obtain, for example, fluorescent and phase-contrast images. The second cells 50 may be stained in step S802 prior to being introduced into the channel, for subsequent imaging of the second cells in step S805 using the imaging device 84. However, it will be appreciated that imaging of the cells need not necessarily be performed. For example, in some studies no imaging may be needed as the collection of cells and their sequencing/propagation may be sufficient for determination of optimal cell candidates.

In step S806 further downstream analysis may be performed. For example, the analysis may include determining an avidity between at least one of the second cells and the first cells based on the applied shear stress at which the at least one second cell detached. Alternatively, or additionally, the detached second cells may be propagated (for example, left to proliferate before subsequent injection into a patient). Further examples of downstream analysis are provided in more detail in the ‘further details and experimental validation’ section below.

Alternatively, or in addition, the second cells that remained attached to the first cells 51 in the channel 12 may be further analysed. For example, the remaining cells adhered inside the device may be quantified using an inverted fluorescent microscope (Nikon Eclipse Ti2-E) that is capable of capturing a large image acquisition of the device substrate containing the remaining cells. Post-analysis using NIS-Elements Advanced Research software (Nikon) with applied thresholding, size and circularity constraints allows for quantification of cell attachment after each shear condition. In other studies no imaging may be needed as the collection of cells and their sequencing/propagation may be sufficient for determination of optimal TCRs or CAR-T cells.

### Further Details and Experimental Validation

### Cell Culture

Me290 (HLA-A2⁺/NY-ESO-1⁺) and NA8 (HLA-A2⁺/NY-ESO-1⁻) human melanoma cells; and SupT1 human T cells were maintained in RPMI 1640 medium. B16F10 (gp100⁺) mouse melanoma cells were maintained in DMEM, high glucose, GlutaMAX™ medium. All culture media was supplemented with 10% (v/v) FBS and 1% (v/v) penicillin/streptomycin. All cell lines were cultured at 37° C./5% CO₂ and passaged every 2-3 days using 0.25% trypsin-EDTA and used at passage numbers 20 or lower. Cell lines were validated free of mycoplasma contamination monthly.

### TCR Transduction

Full-length codon-optimized T cell receptor (TCR) (AV23.1/BV13.1) chain sequences of a dominant NY-ESO-1⁺ specific T cell clone of patient LAU 155 were cloned in the pRRL third-generation lentiviral vectors as an hPGK-AV23.1-IRESBV13.1 construct. Structure-based amino acid substitutions were introduced into the wild-type TCR sequence, as previously described. This generated a panel of TCR-transduced SupT1 T cell variants of varying avidity to the NY-ESO-1⁺ antigen: non-transduced (NT, weak avidity), wild-type (WT, intermediate avidity) and double-mutant beta (DMP, high avidity). Lentiviral production was performed using the standard calcium-phosphate method and concentrated supernatant of lentiviral-transfected 293T cells was used to infect TCRα knockout CD8+ SupT1 T cells. Levels of transduced TCR expression were measured using flow cytometry with PE-labelled HLA-A*0201/NY-ESO-1 specific multimers and FITC-conjugated BV13.1 antibody (Beckman Coulter, USA).

### Primary Mouse T Cell Culture and Activation

TCR-transgenic Thy1.1+ pmel-1 (Pmel) mice and Thy1.2+C57Bl/6 mice were originally purchased from The Jackson Laboratory (Bar Harbor, ME, USA) and maintained in the École Polytechnique Fédeŕale de Lausanne (EPFL)-CPG animal facility. The genotyping was confirmed by Transnetyx (Cordova, TN, USA).

Experimental procedures in mouse studies were approved by the Swiss authorities (Canton of Vaud, animal protocol ID 3206) and performed in accordance with EPFL-CPG guidelines. Spleens from Pmel or 57B1/6 mice were mechanically disrupted and ground through a 70-μm strainer. Red blood cells (RBC) were lysed with ACK lysis buffer (2 mL per spleen) for 5 min at 25° C. The splenocytes were washed twice with PBS which contained RPMI-1640, FBS (10% v/v), HEPES (1% v/v), penicillin/streptomycin (1% v/v), and β-mercaptoethanol (0.1% v/v). Splenocytes were then resuspended at a cell density of 2×10⁶ cells/mL in complete RPMI medium supplemented with mouse IL-2 (10 ng/mL, PeproTech, UK) and IL-7 (2 ng/mL, PeproTech), as well as human gp100 (1 μM, GenScript) or a CD3/28 T cell activation kit (Miltenyi Biotec) for Pmel or C57Bl/6 splenocytes, respectively. After 3-day culture, live cells were enriched by density gradient centrifugation (Ficoll-Paque PLUS, GE Healthcare, UK), followed by 2-day culture at a cell density of 0.5-1.0×10⁶ cells/mL in complete RPMI medium supplemented with mouse IL-2 (10 ng/mL) and IL-7 (10 ng/mL) to obtain activated CD8+ T-cells with purity >95% validated by flow cytometry (as described above).

### Microfluidic Device Design and Fabrication

Photolithographic masks were printed out on fine grain emulsion film (Micro Lithography Services, UK). Master moulds were fabricated using standard soft photolithography techniques in a class 1000 cleanroom (Imperial College London, UK). SU-8 (GM1070 series, Gersteltec, Switzerland) negative photoresist was spin coated onto 3-inch plasma treated silicon wafers (Siegert Wafer, Germany) at 950 rpm for 45 s to obtain 100 μm high features. Wafers were baked at 65° C. and 100° C. for 2 min and 10 min, respectively. With reference to FIG. 1, resist-coated wafers were then patterned using UV photolithography with a mask aligner (Karl Suss MJB-3, Suss MicroTec, Germany) to define masters containing shear devices 100 (also referred to as microfluidic devices throughout the present disclosure) with a geometry of 1×0.1×50 mm (width by height by length) coupled with 2 mm wide fluidic inlets and outlets. The master wafer was completed through a series of post-bakes at 65° C. and 100° C. for 2 min and 10 min, respectively. This was followed by a 10 min SU-8 development step using Propylene glycol methyl ether acetate (PGMEA, Sigma-Aldrich, UK) and a 10 min hard-bake at 100° C. after washing with iso-propyl alcohol. The SU-8 features were verified to be within +10% of the target height set using a surface profilometer (AlphaStep 200, KLA-Tencor, UK). Polydimethylsiloxane (PDMS, Dow Corning, UK) mixed with its cross-linker at a 10:1 ratio (wt/wt) was poured onto silicon master moulds, degassed for 1 h using a vacuum desiccator, and cured at 65° C. for 24 h in an oven. Cured PDMS devices were removed from the moulds using a scalpel and punched using a 2-mm biopsy punch (Agar Scientific, UK) to obtain fluidic inlets and outlets. Punched PDMS devices and glass microscope slides were bonded together after plasma treatment (Emitech K1050X, Quorum Technologies Ltd, UK) at 300 mmTor O₂ at 50 W for 1 min and transferred to a hot plate for 10 min at 100° C.

### Melanoma Cell Seeding

Devices were handled using aseptic techniques and sterilised with 70% ethanol for 1 min followed by washing with sterile phosphate-buffered saline (PBS). Devices were then coated with poly-L-lysine (0.01%, Sigma-Aldrich) to improve Me290 melanoma attachment or fibronectin (10 μg/ml, Sigma-Aldrich) to improve NA8 and B16F10 cell adhesion. All coatings were left for 1 h at 37° C./5% CO₂ followed by 3× sterile PBS washes. Melanoma cells were detached from culture flasks (as described above) and 10 μL of cell suspensions concentrated to 4×10⁶ cells/mL were fed into each device. Cells were permitted to adhere to devices for 15 min, followed by feeding with 500 μL of complete RMPI medium for overnight incubation (37° C./5% CO₂). Melanoma cells were cultured in the device for 1-2 days until ˜95% confluence. Devices were fed daily with fresh media. Adhesion experiments were conducted on melanoma cells stained with calcein-AM (5 μM, ThermoFisher) and Hoescht (1 μM, ThermoFisher) for 40 min at 37° C./5% CO₂ followed by 3×PBS washes. With reference to FIG. 8, shear stress was induced by adjusting the flow rates of DMEM media infused into the device 100 via TYGON® inlet tubing (0.76 mm ID, VWR) using a programmable syringe pump 80 (PHD ULTRA™, Harvard Apparatus, UK). Flow rates were ramped up from 0 μL/min to the set experimental value for 5 s and kept at a constant flow for 50 s before ramping down to 0 μL/min.

Shear stresses examined were set as the following: 0, 1, 1.9, 3.8, 5.7, 7.7, 11.5, 14.4, 19.2 Pa. Fluorescent and phase-contrast images of the 1×50-mm channel 12 at a height of 3 μm were recorded using a 10× objective. All live-cell imaging was performed on a Nikon Eclipse Ti2 Inverted Microscope 84 (Nikon, Japan) with an incubated humidified chamber (Okolab, Italy) at 37° C./5% CO₂. The microscope was attached with a monochrome Nikon DS-Qi2 camera and LED illuminator for phase-contrast and fluorescent imaging. Filters were set as the following: DAPI (excitation 358 nm/emission 461 nm), FITC (excitation 490 nm/emission 525 nm) and Cy5 (excitation 649 nm/emission 665 nm).

### Melanoma Cell Viability

Devices with a confluent monolayer (˜95%) of melanoma cells were stained with propidium iodide (1 μM, ThermoFisher), a red-fluorescent nuclear and chromosome counterstain used to identify dead cells. Hoescht solution (1 μM) was used as a nuclear stain to determine total percent viability. Monolayers were transferred to a fluorescent microscope 84 and attached to a syringe-driven pump 80 via the inlet reservoir. Staining media was loaded into the syringe and shear stresses from 0-19.2 Pa were tested by adjusting input flow rates. The monolayer experienced shear-induced flow for 1 minute per flow rate examined and images were acquired after each flow rate tested. Thresholding and size constraint analysis were used to quantify percent cell viability as a function of shear stress using the following equation:

$$\% cell viability = 100 \times \frac{total number of cells - dead cells}{total number of cells}$$

### T Cell Selection by Cellular Avidity

SupT1 human T cells and primary mouse T cells were labelled with calcein-AM (5 μM, 1×10⁶ cells/ml) for 1 h at 37° C./5% CO₂ followed by 3×PBS washes. SupT1 DMP and primary Pmel T-cells were stained with calcein green; SupT1 wild-type (WT) T cells were stained with calcein red; SupT1 non-transduced (NT) and primary B6 T cells were stained with calcein blue. The devices containing attached melanoma cells were transferred to the microscope stage and incubated (37° C./5% CO₂) with 1% bovine serum albumin (BSA, Sigma-Aldrich) for 1 h to block nonspecific T-cell adhesion. SupT1 T cell experiments were initiated by mixing DMP:WT:NT T cells at 1:1:1 ratios before infusion into Me290 (NY-ESO-1⁺) or NA8 (NY-ESO-1⁻) coated devices using a pipette tip followed by inserting inlet and outlet tubing (0.76 mm ID, VWR) connected to the syringe pump and collection wells, respectively. For primary mouse experiments, B16F10 melanoma cells were treated with 1 μM hgp100 peptide (GenScript, US) 30 mins prior to T cell infusion. Pmel:B6 T cells were mixed at 1:1 ratios and fed into the device using the same method described previously. T cells were left to attach to melanoma cells for 10 min under static conditions (i.e. without flow of fluid through the channel 12 to apply a shear stress). T cell detachment and collection was examined under shear-induced flow in the range of 0-19.2 Pa using a programmable syringe pump 80, as previously described. Fluorescent and phase-contrast images were recorded after each shear stress tested using the large-image acquisition feature on NIS-Elements Advanced software (Nikon). Device images and collection wells 81 were captured with a scan area of 40×2 fields and 4×4 fields, respectively, and stitched together with 15% overlap. Collection wells were transferred to an incubator where T cells were left to proliferate overnight for further downstream analyses.

### T Cell Activation

SupT1 human T cells at 10⁶ cells/mL were stained using a Fluo-8 calcium flux assay kit (5 μM, Thermo Fisher Scientific) in serum free DMEM media for 60 min at 37° C./5% CO₂ followed by 3×PBS washes. T cells were resuspended in complete DMEM media and 10 μL of cell suspension was fed into the device coated with confluent monolayers of Me290 or NA8 under the microscope 84 (37° C./5% CO₂).

Fluorescent and phase-contrast time-lapse images were captured at the middle of the channel with a 1×1 mm field of view and taken every 2 s for 10 min under static conditions.

### IFN-γ Secretion

Fibronectin pre-treated microfluidic devices 100 were seeded with 12,500 B16F10 melanoma cells and left to attach for 1 h at 37 C/5% CO₂ followed by 1 μM hgp100 peptide simulation 30 mins prior to T cell introduction. 25,000 Pmel or WT T cells (2:1, effector:target ratio) were suspended in modified RPMI media supplemented with IL-2 and IL-7 and introduced into the device. The co-cultured devices were incubated overnight and conditioned media was collected after 24 h from the outlet reservoir. The collected media was centrifuged and 50 μL of the cell-free supernatants were removed for analysis. The IFN-γ levels were evaluated by using a highly sensitive mouse IFN-γ specific enzyme-linked immunosorbent assay (ELISA) kit (LEGEND MAX™, BioLegened, UK) according to the manufacturer's instructions. Absorbance values for standards and samples were recorded at 450 nm using a microplate reader (Infinite F50, Tecan, Switzerland).

### T Cell Cytotoxic Functionality

Primary mouse T cells collected in wells 81 from shear stress experiments were split into 3-fractions based on cellular avidity to B16F10 melanoma cells: strong collected at 11.5-19.2 Pa, medium at 3.8-7.7 Pa and weak at 1.9 Pa. Each fraction was tested for its ability to lyse melanoma cells using a non-radioactive cytotoxicity assay (CytoTox 96@ kit, Promega Corporation) by the release of lactate dehydrogenase (LDH) and its conversion into red formazan product. B16F10 tumour cells were incubated at 2000 cells/well in non-treated 96-well round bottom plates with T cell fractions collected from the shear device product outlet 12. Each T cell fraction was diluted to 20 cells/well to match the limited number of T cells collected under the highest shear fraction (14.4-19.2 Pa). The plate was then incubated for 8 h at 37° C./5% CO₂. Spontaneous release of LDH in T cells and tumour cells were measured as well as the culture media background in separate control wells. All samples were run in triplicate and made up to a total volume of 100 μL/well in phenol red-free RPMI supplemented with 10% FBS, 1% penicillin/streptomycin, 10 ng/ml IL-2 and IL-7. The maximum LDH release by tumour cells was measured by adding 10 μL of lysis solution (10×) to yield complete lysis. At the end of the incubation period, the well plate was centrifuged and 50 μL of supernatant from each well was transferred into a fresh flat bottom 96-well plate and mixed with 50 μL/well of LDH CytoTox 96 reagent. The plate was light protected and incubated at room temperature for additional 30 min, and the reaction was stopped by the addition of the stop solution. Absorbance data at 492 nm was measured using a 96-well plate reader (Infinite F50, Tecan) alongside T cell-free and melanoma-free controls. The percentage of specific lysis was calculated using the following equation:

$$\frac{Experimental release - B 16 F 10 spontaneous release - T cell spontaneous release}{B 16 F 10 maximum release - B 16 F 10 spontaneous release} \times 100$$

### Immunofluorescence Staining

Microfluidic devices 100 were plasma bonded to glass cover slips (25×60-mm). Devices were treated with 70% ethanol followed by 3×PBS washes and incubated for 1 h with fibronectin (10 μg/mL). 12,500 B16F10 melanoma cells were seeded into the device and left to adhere for 1 h at 37° C./5% CO₂ followed by treatment with 1 μM hgp100 peptide 30 mins prior to T cell introduction. With reference to FIGS. 5, 8 and 20, primary mouse Pmel (210a) or B6 (210b) T cells were fed into the device 100 at a 2:1 ratio (effector:target cells) and left to bind to the melanoma monolayer 51 for 10 min. High (14.4 Pa), low (1.9 Pa) and no (0 Pa) shear stress conditions were induced using a programmable syringe pump 80. Cell conjugates were fixed with 4% paraformaldehyde for 20 min, blocked and permeabilized with 1% BSA and 0.5% saponin (all Sigma-Aldrich). Cells were then incubated with primary antibodies (Anti-Connexin 43/GJA1 antibody, abcam, UK) 1/200 diluted in 1% BSA in PBST (PBS+0.1% Tween 20) overnight at 4° C., then washed 3× with PBS. Cells were then treated with secondary antibodies (Goat Anti-Rabbit IgG, Alexa Fluor® 488, abcam) and Phalloidin (Phalloidin-iFluor 594 Reagent, abcam) 1/200 diluted in 1% BSA for 1 h in the dark. The immunological synapse for 20 cell conjugates per condition were captured using a confocal microscope 84 (Nikon Eclipse Ti2).

### Image Processing and Statistical Analysis

NIS-Elements Advanced software (Nikon) was used for image processing and analysis. Large-image acquisitions of devices were cropped to 25×0.8 mm (length by width) so that cells located 0.1 mm either side of the channel walls were excluded from the analysis due to experiencing non-uniform shear stress. The object count tool was used to quantify T cells based on thresholding with applied size and circularity constraints. The number of T cells counted for each condition ranged from 0-3000 cells for all experiments. Fluid dynamic simulation of shear stress in the device was modelled using laminar flow physics and the Navier-Stokes equation describing the motion of Newtonian fluids. In each simulation, a no slip boundary condition was used and inlet velocities were inputted based on experimental flow rates tested. Shear stress plots were measured at a z-plane of 4 μm across the channel width (x-plane) to represent the height of the T cell-to-tumour cell interaction across the device. Activation was analysed using a custom MATLAB (MathWorks, USA) script to determine active versus inactive T cells. The criterion for an active T cell was characterised by the standard deviation divided by the mean intensity using a threshold value of 0.2. The accumulation of Cx43 at the synapse was quantified using ImageJ software by measuring the ratio of the mean grey values at the synapse and opposite end of the T cell. Prism software (GraphPad, Inc, USA) was used to present graphical data and for statistical analysis. Each experiment was performed independently and repeated three times for each condition. One-way or two-way ANOVA plus Bonferroni post-tests were used to calculate the difference between means, with p-values less than 0.05 considered significant. Single asterisk show *p<0.05, double asterisk show **p<0.01, triple asterisk show ***p<0.001. Data is presented as means, with error bars set as the standard error of the mean (SEM) unless stated otherwise.

### Results

Microfluidic shear devices 100, illustrated in FIG. 1, were designed to collect T cells based on their cellular avidity to tumour cells 51. The main component of these devices is T cell-tumour cell interaction region that consists of a 1.0×0.1×50-mm (W×H×L) straight microchannel 12 on which T cells 50 interact with adherent tumour cells 51. An interaction between a T cell 12 and a tumour cell 51 is illustrated schematically in FIG. 6, which shows the CD8 62 and TCR 60 of the T cell and the MHC 61 of the tumour cell 51. The above geometry of the channel 12 allows interacting cells to experience near identical shear stress (±1%) at a given flow rate to be collected at the product outlet 12. In contrast, cells located 0.1 mm or less from either side wall of the channel experienced non-uniform shear and were diverted to a waste outlet 14, as illustrated in more detail in FIG. 2. As illustrated in FIGS. 7a to 7d, by adjusting the flow rate and thus applied shear stresses, the device 100 can discriminate between T cells 50 of varying cellular avidities, which can be easily sorted downstream for subsequent processing and/or downstream analysis of the detached T cells.

### Tumour Cell Monolayers Remained Attached Under Shear

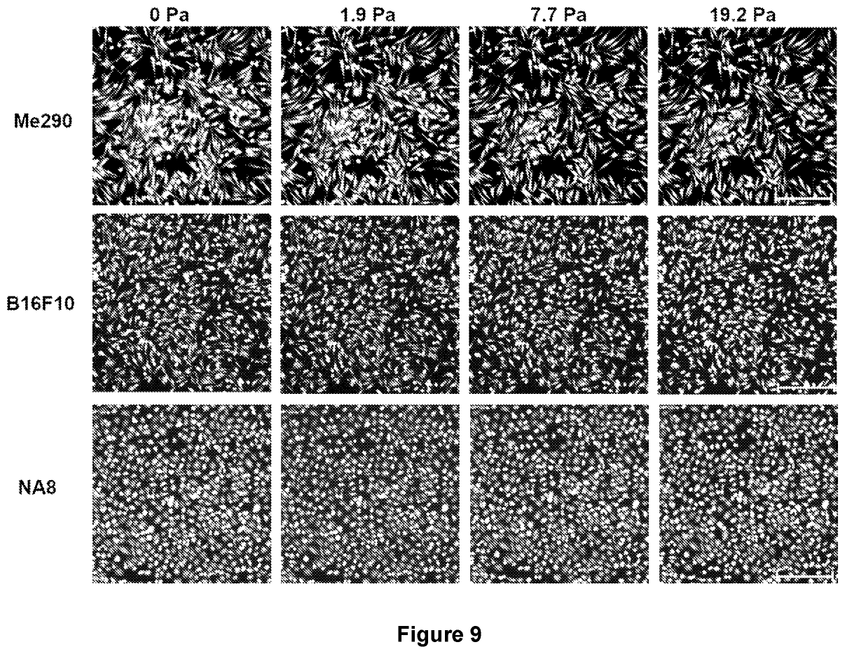

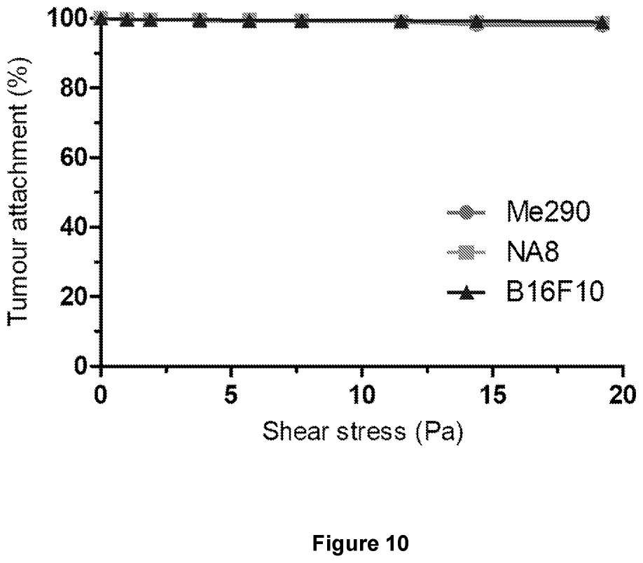

To assess the adhesion of tumour monolayers 51 under shear, devices 100 seeded with Me290, NA8 and B16F10 melanoma cells were subjected to ramping shear stresses from 0-19.2 Pa, as illustrated in FIG. 9, which shows representative images of Me290 (top) and B16F10 cell attachment under 0-19 Pa shear stresses (tumour cells stained with calcein-AM, scale bar: 250 μm). Our data indicates that cells collected under applied shear stresses of 0-1.9 Pa contained poorly adherent cells or those bound non-specifically, and thus this initial shear range can be considered as a wash step. As can be seen in the graph of FIG. 10, which shows quantification of melanoma cell adhesion Me290 (NY-ESO-1⁺), NA8 (NY-ESO-1⁻) and B16F10 (gp100⁺), greater than 99.9% of all three cell types remained attached under 0-11.5 Pa shear stress. At the highest experimental shear stress of 19.2 Pa, 98.1±1.8%, 99.7±0.4% and 98.5±0.6% of Me290, NA8 and B16F10 cells remained attached, respectively.

### Tumour Cell Monolayers Remained Viable Under Shear

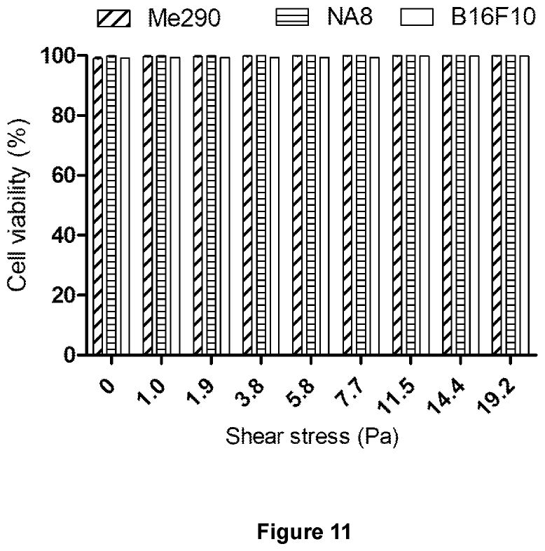

Viability of tumour cells under shear stress were examined using propidium iodide (PI), a nuclear stain for dead cells. This test was to ensure that tumour cell monolayers remained viable in our device 100 under high shear stress so that cell-cell and cell-substrate analyses are not comprised by poor cell viability. Confluent monolayers of Me290, NA8 and B16F10 melanoma cells were subjected to increasing shear stress ranging from 0-19.2 Pa by varying inlet flow rates. Viability data revealed that >99% of tumour cells remained viable even under high shear stress. In fact, as illustrated by the graph of FIG. 11, the percent viability of the tumour monolayer increased, as shear stress increased due to dead cells detaching from the substrate.

Activation of Tumour Cell-Bound SupT1 T cells Correlates with Increasing Avidity

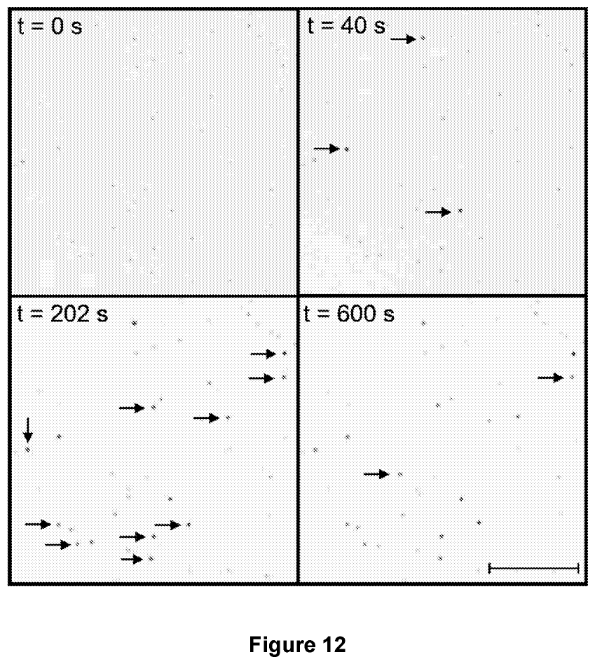

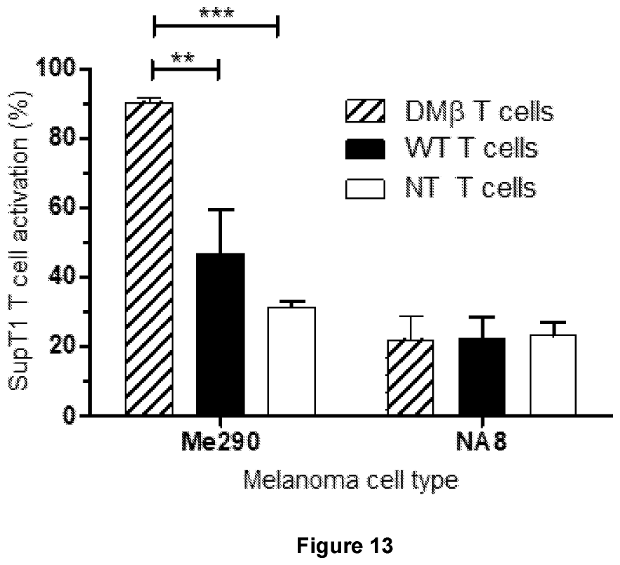

To ensure that T cell binding resulted in physiological T cell activation, we probed the concentrations of intracellular calcium, a critical signalling step in T cell activation, in bound TCR variants using fluorescent dyes. SupT1 human T cell variants with TCRs of varying avidities to the NY-ESO-1 antigen were used to validate activation in our device. Unmodified wild-type (WT, k_{off}=4.08×10⁻²/s) represented a normal physiological range, Non-transduced (NT, k_{off}=n/a) represented non-reactive TCRs and double mutant beta (DMP, k_{off}=0.78×10⁻²/s) represented high avidity TCRs. SupT1 T cells were stained with a calcium binding dye and then infused into devices. T cells bound to Me290 device monolayers showed activation that corresponded to their relative avidities as determined by analysing the calcium flux, as illustrated by the image shown in FIG. 12 which shows representative calcium flux time-lapse images of DMP T cell activation on Me290 melanoma monolayer (white arrows indicate T cells that activate, scale bar: 250 μm). As illustrated in the graph of FIG. 13, which shows quantification of SupT1 T cell activation on Me290, NA8 and monolayer-free devices, automated MATLAB scripts revealed that the percentage of SupT1 T cell activation increased, as the avidity to the NY-ESO-1 antigen increased. DMP T cells exhibited activation of 90.3±1.2% on Me290s, whereas WT and NT T cells recorded significantly lower (p<0.01) activation of 46.7±12.6% and 31.3±1.7%, respectively. These results validated that avidity influenced initial signalling events that lead to T cell activation. Moreover, SupT1 T cells in contact with NA8 cells (NY-ESO-1\hat{ }⁻) exhibited significantly less activation compared to Me290 cells (NY-ESO-1\hat{ }⁺). This validated that specific antigen recognition was required for T cell activation.

### SupT1 T Cells can be Collected Based on Cellular Avidity

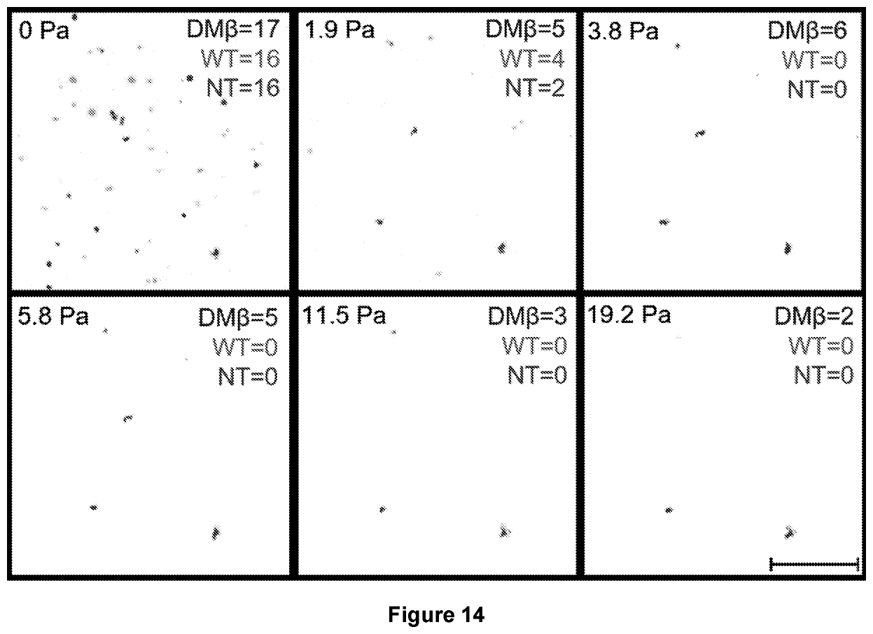

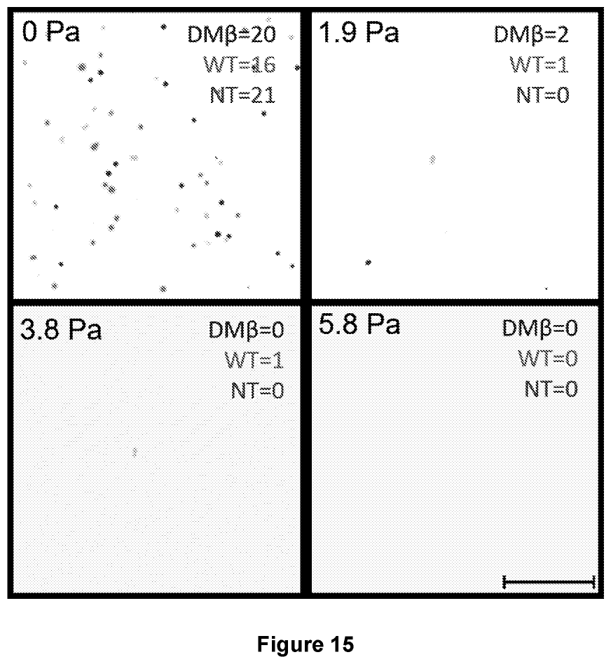

SupT1 T cell variants dyed with calcein variants were infused into devices seeded with monolayers 51 of Me290 (NY-ESO-1⁺) and NA8 (NY-ESO-1⁻) melanoma cells, as illustrated by the images shown in FIG. 14 (which shows representative images of SupT1 T cell detachment from Me290 melanoma cells (NY-ESO-1\hat{ }⁺), scale bar: 250 μm) and FIG. 15 (which shows representative images of SupT1 T cell detachment from NA8 melanoma cells (NY-ESO-1\hat{ }⁻), scale bar: 250 μm), and subjected to 0-19.2 Pa shear stresses at 37° C./5% CO₂. Automated image analyses were conducted on 0-3000 SupT1 T cells per condition bound to 20 mm² per device 100 to determine cellular avidity.

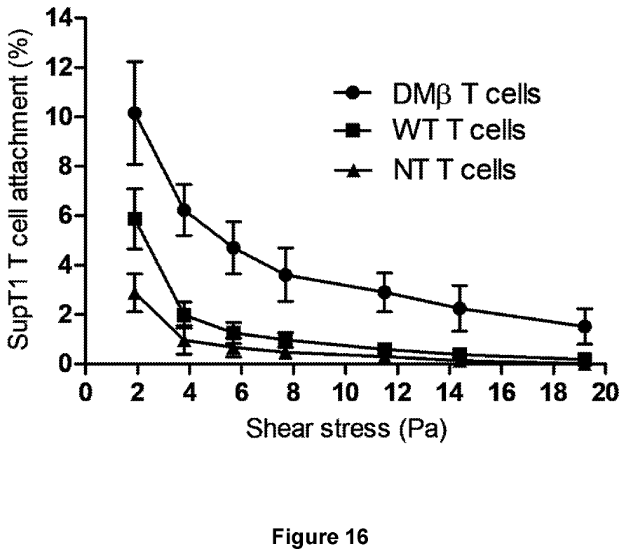

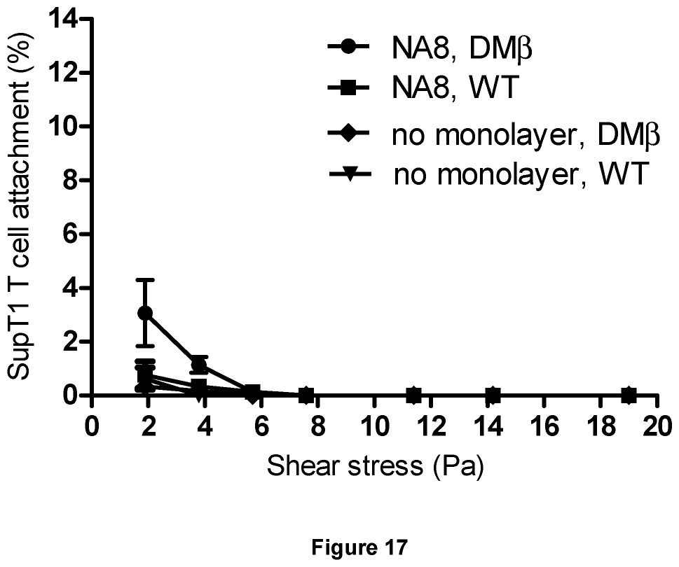

As expected, the adhesion of SupT1 T cells followed their relative avidities to the NY-ESO-1 antigen. As illustrated in the graph of FIG. 16, which shows quantification of SupT1 T cells with varying avidities (DMP with high avidity, WT with intermediate avidity and NT with weak avidity) to attached Me290 cells (NY-ESO-1\hat{ }⁺) under shear-induced flow, DMP T cells with high avidity remained more strongly adhered compared to WT and NT T cells, as shear stress was increased from 1.9-19.2 Pa. Notably, 1.5±0.7% of DMP T cells remained adhered at 19.2 Pa; whereas, 99.8±0.1% and 100% of WT and NT T cells were detached, respectively. In contrast, as illustrated in the graph of FIG. 17, which shows quantification of SupT1 T cell attachment to NA8 melanoma cells (NY-ESO-1\hat{ }⁻) and monolayer-free devices, SupT1 T cells displayed weaker adhesion to NA8 (NY-ESO-1\hat{ }⁻) melanoma cells and monolayer-free bare devices. Under these conditions, all SupT1 T cell variants were detached by 7.7 Pa and thus, this verified that cellular avidity was dominated by the recognition of the specific NY-ESO-1 antigen.

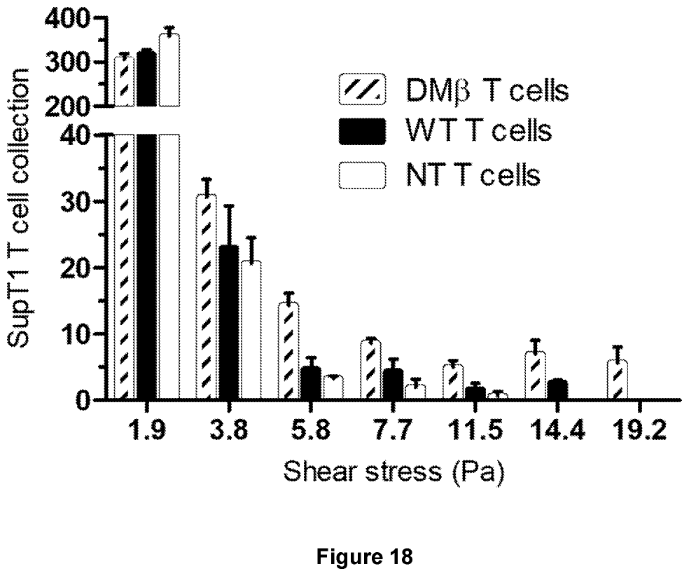

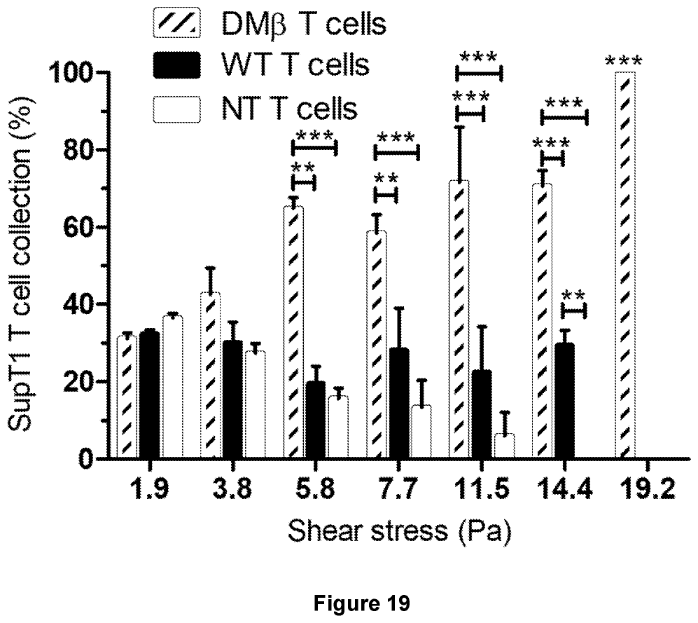

Effluents of devices under 1.9-19.2 Pa shear stresses were then collected and the numbers of SupT1 T cell variants quantified using NIS-Elements Advanced software, as illustrated by the graphs shown in FIG. 18 (which shows quantification of SupT1 T cells collected from the product outlet of the device under increasing shear stress) and FIG. 19 (which shows the percentage of each SupT1 T cell variant collected). There was no statistical difference (p >0.05) among the T cell purities collected under shear stresses of 1.9-3.8 Pa. However, fractions collected under shear stresses of 5.7-19.2 Pa exhibited a statistically significant higher purity of DMP T cells in comparison to WT and NT T cells. Importantly, at 19.2 Pa 100% of collected fractions contained only DMP T cells. These results correlated with the data obtained from T cell attachment experiments described above T cells with stronger cellular avidities were therefore successfully isolated from weaker avidity counterparts.

### Primary T Cells can be Collected Based on Cellular Avidity

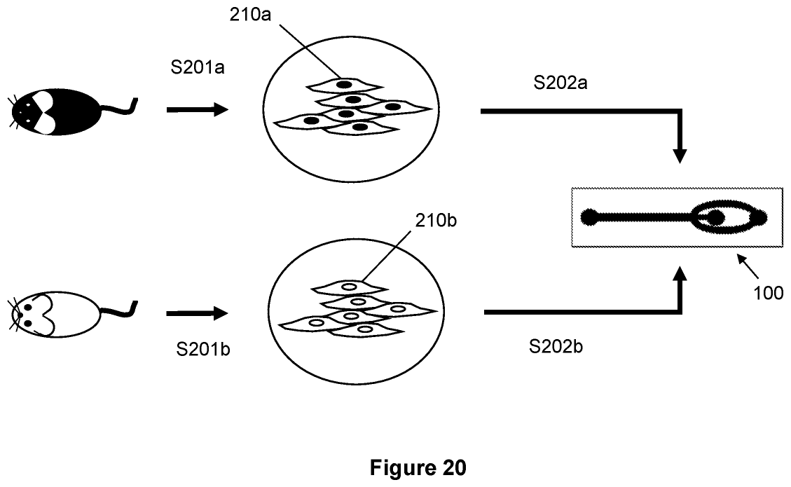

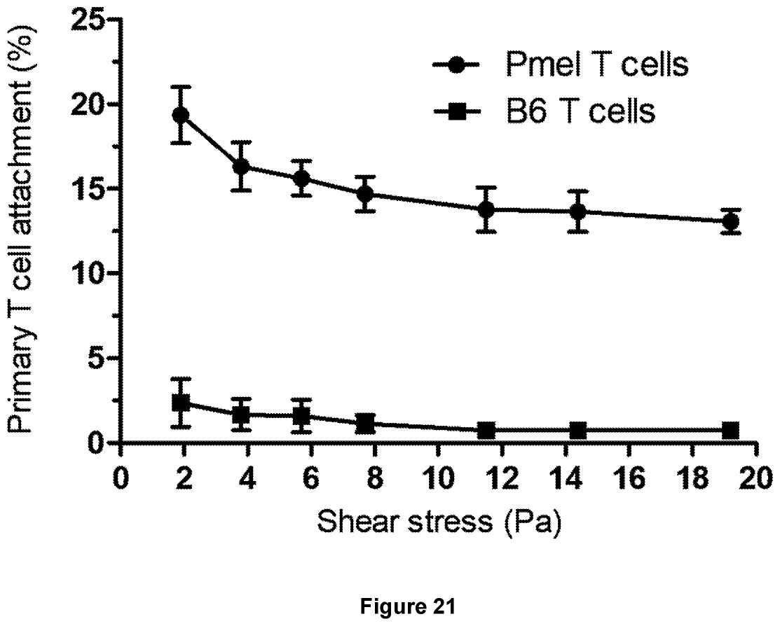

With reference to FIG. 20, which shows a schematic diagram of mouse T-cell isolation, expansion and seeding, we next investigated if primary mouse T cells could be isolated based on their cellular avidity to gp100 antigens presented on the surface of B16F10 melanoma cells. Pmel (high avidity) 210a and B6 (normal physiological avidity) 210b T cells were harvested (steps S201a and S201b) from mice according to institutionally approved protocols and infused (steps S202a and S202b) into the device 100 coated with adhered B16F10 cells. As illustrated in the graph of FIG. 21, which shows quantification of Pmel (high avidity) and B6 T cells (weak avidity) attachment to B16F10 melanoma cells (gp100\hat{ }⁺) under increasing shear stress, Pmel T cells 210a bound more strongly to B16F10 cells compared to B6 T cells 210b, as shear stress was increased. Pmel T cells 210a were differentiated from B6 T cells 210b even at low shear stresses of 1.9 Pa, with 19.4±1.7% and 2.4±1.4% of Pmel 210a and B6 210b T cells that remained bound to the tumour monolayer 51, respectively. Data gathered here demonstrated that primary mouse T cells bind more strongly to the specific target antigen, as avidity increases, which correlated with results obtained previously.

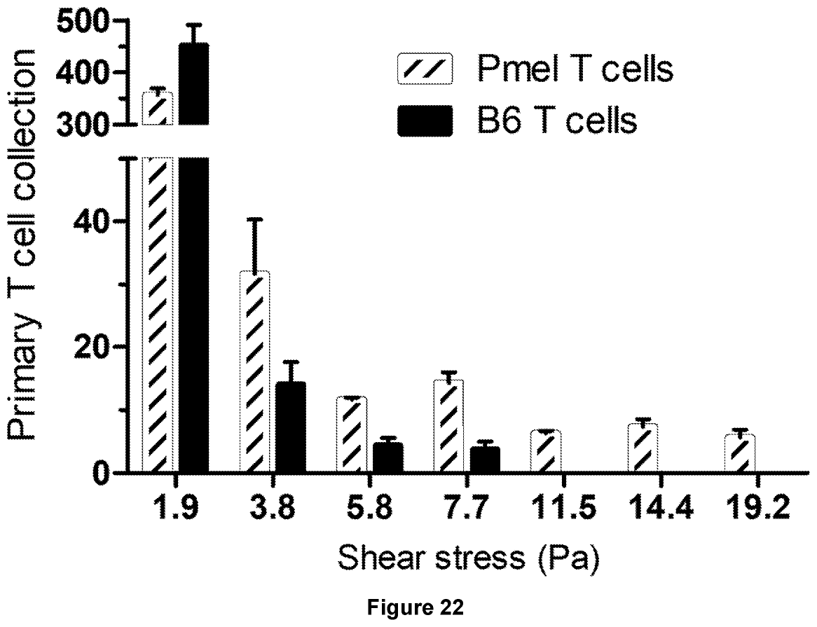

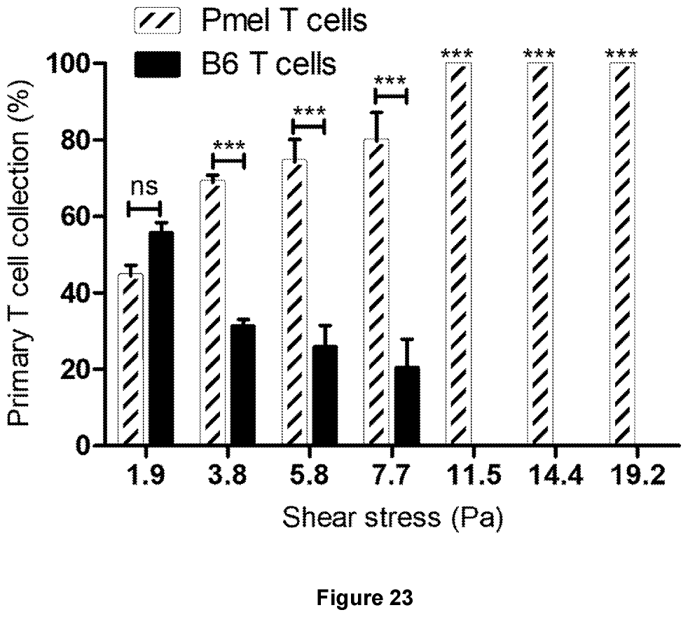

As illustrated by the graphs shown in FIG. 22 (which shows quantification of primary mouse T cell collection from the product outlet under increasing shear stress) and FIG. 23 (which shows percentage of primary mouse T cells collected), primary mouse T cells were then collected from the product outlet 12 and quantified in order to determine the percentage of Pmel 210a and B6 210b T cells collected after each shear stress tested. We observed that Pmel 210a and B6 210b T cells collected were not significantly different (p >0.05) after 1.9 Pa. In contrast, we analysed statistically different (p<0.001) percentages of Pmel 210a and B6 201b T cells collected when we increased shear stress to 3.8 Pa. Notably, at shear stresses 11.5 Pa, 100% pure Pmel 210a T cell fractions were successfully isolated from weaker B6 210b T cells, indicating that primary mammalian cells can be successfully isolated based on their cellular avidity.

Primary T cell Fractions with High Cellular Avidity Have Greater Cytotoxic Efficiencies

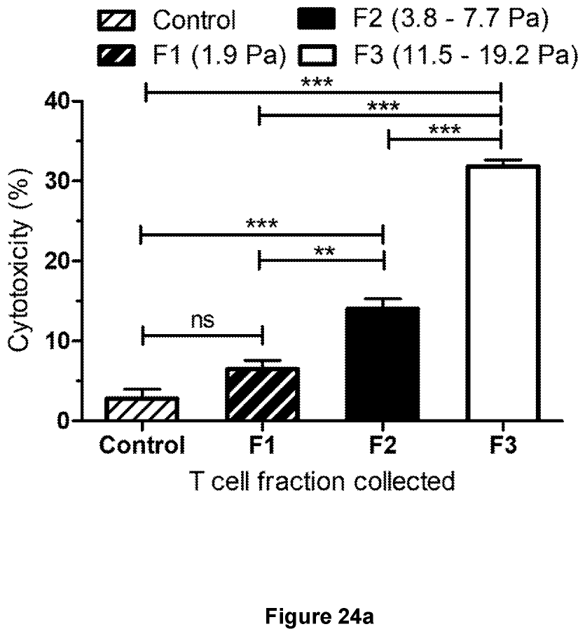

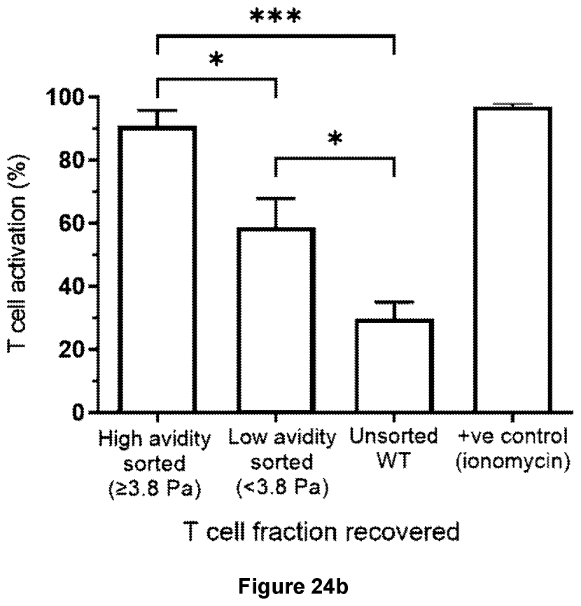

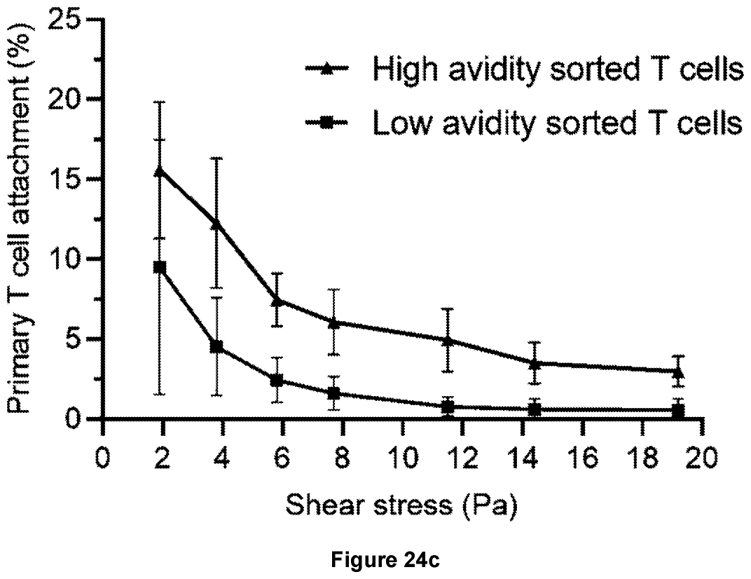

To investigate if T cells with superior anti-tumour capabilities could be collected with this technology, we collected primary mouse T cells under sterile conditions. T cells were collected in 3-fractions based on their cellular avidity to the tumour monolayer 51 (as described above): strong collected at 11.5-19.2 Pa, medium at 3.8-7.7 Pa and weak at 1.9 Pa. Each fraction was examined for its ability to lyse B16F10 melanoma cells by analysing absorbance data obtained from cytotoxicity tests. As expected, the numbers of tumour cells lysed correlated with the cellular avidity to the gp100 antigen, as illustrated by the graph of FIG. 24a, which shows cytotoxicity of primary T cell fractions collected from the device outlet based on their cellular avidity to B16F10 melanoma cells: F1 (1.9 Pa), F2 (3.8-7.7 Pa), and F3 (11.5-19.2 Pa). The strongest cellular avidity T cell fraction collected under 11.5-19.2 Pa induced the most potent anti-tumour response in vitro, with 31.2±0.8% of tumour cells lysed. In contrast, significantly fewer tumour cells were lysed with medium and weak T cell fractions, which exhibited 14±1.2% and 6.5±1.1% cytotoxicity, respectively.

To explore persistence of the high avidity phenotype, we sorted T cells collected from devices into two fractions: those collected after application of “high” shear (3.8 Pa) or “low shear” (<3.8 Pa). Cells were cultured for 24 hours in fresh medium at 37° C./5% CO2 and then stained with a Fluo-8 calcium flux indicator dye before being transferred onto new devices with fresh B16F10 tumor monolayers. FIG. 24b shows quantification of T cell activation by calcium flux indicator from sorted and unsorted T cell fractions on B16F10 melanoma monolayers. As illustrated in FIG. 24b, our results indicate that T cells recovered with high cellular avidities showed significantly greater activation (90.9±4.9%) compared to T cells recovered with low avidities (58.9±8.9%) and an unsorted (29.8±5.3%) WT mixed population.

We then examined if the binding characteristics of primary T cells collected based on cellular avidity persisted when exposed to fresh tumor monolayers. Primary T cell fractions were sorted into high and low shear fractions as described above, cultured for 8 hours, then differentially labelled with green and red calcein-AM dyes respectively, mixed together and fed into new devices coated with fresh tumor monolayers. FIG. 24c shows quantification of persistence of cellular avidity after second device challenge. Primary T cell binding to B16F10 monolayer devices from high and low avidity T cell fractions recovered under increasing shear stress. Data are represented as mean±SEM error bars, N=3, P-values are calculated using one-way or two-way ANOVA with Bonferroni's multiple comparisons corrections: *P<0.05,**P<0.01, ***P<0.001. As illustrated in FIG. 24c, high cellular avidity primary T cells from devices maintained significantly stronger avidity to fresh B16F10 tumor monolayers compared to their low cellular avidity counterparts at all tested shear challenges from 1.9 to 19.2 Pa. These results validated that primary T cells selected based on higher cellular avidities maintained their phenotypic behaviors including cytotoxicity, T cell activation and binding after recovery and culture.

Cytokine Secretion of Primary T Cells Correlates with Increasing Avidity

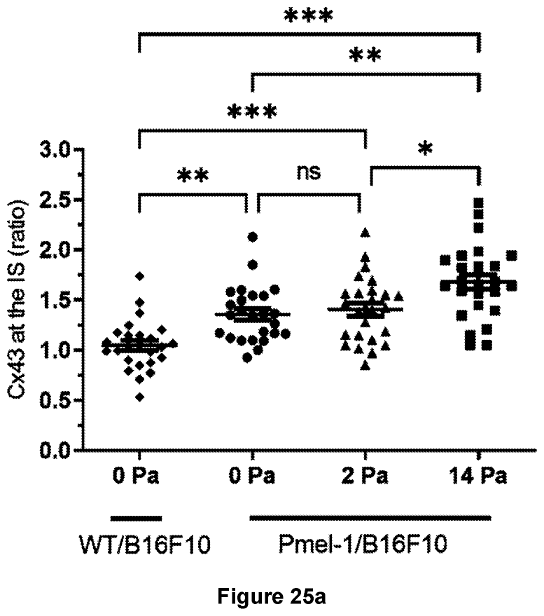

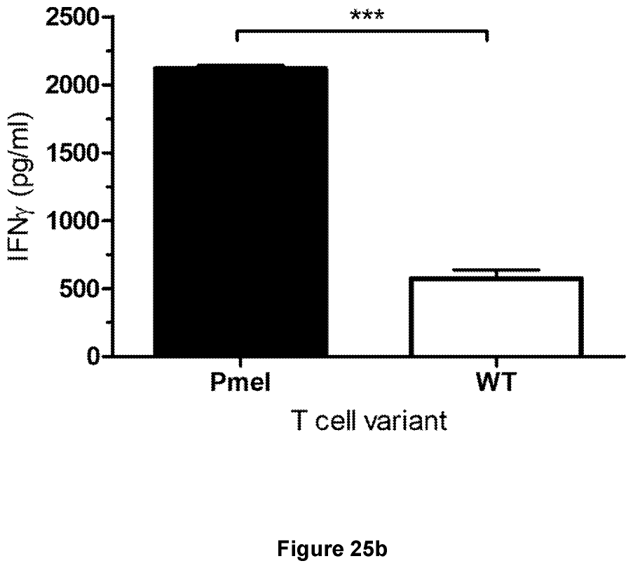

Whether differences in IS formation were observable in cells bound at different shear stresses was examined. Previous studies revealed that the gap junction protein Cx43 accumulates at the IS in an antigen-dependent manner. Accumulation of Connexin-43 (Cx43) gap junction proteins and F-actin at the IS under high (14.4 Pa), low (1.9 Pa) and no shear stress conditions using immunofluorescence staining with confocal microscopy was observed. FIG. 25a shows quantification of the mean fluorescent intensity ratio of Cx43 accumulation at the immunological synapse to the opposite site after high, low and no shear-induced flow conditions, with 25 cell conjugates analyzed per condition. As illustrated in FIG. 25a, by measuring the mean fluorescence intensity of Cx43 at the synapse it was found that antigen-specific Pmel T cells formed a stronger IS with B16F10 tumor cells compared to WT T cells at 0 Pa. Moreover, it was found that Pmel T cells that remained attached to B16F10 cells after high shear stress expressed significantly higher levels of Cx43 accumulation, with 17% and 24% increases in fluorescent intensity compared to low and no shear conditions, respectively.

T cell functionality was evaluated by analysing IFN-γ secretion from media collected downstream from the outlet reservoirs of devices co-cultured overnight with a 2:1 ratio of primary T cells to melanoma cells. As illustrated by the graph shown in FIG. 25b, which shows IFN-γ secretion of T cell-tumour cell co-culture media collected at the device outlet, Pmel 210a T cells with high avidity to the gp100 antigen presented on B16F10 melanoma cells released significantly higher levels of IFN-γ at 2120±11.55 μg/ml compared to wild-type B6 210b T cells at 573.3±37.12 μg/ml.

Primary T Cells with High Cellular Avidity Express Higher Levels of Connexin-43 Accumulation at the IS

The cellular avidity takes into account the strength of the IS formation between T cell-tumour cell conjugates. We found that under static conditions Connexin-43 (Cx43) and F-actin accumulates at the cell-to-cell contact site between Pmel-1 210a T cells and B16F10 melanoma cells, which correlates to stable IS formation. Whereas, B6 210b wild-type T cells formed weak synapses with B16F10 melanoma cells, as expected. Moreover, Pmel 210a T cells that remained attached to B16F10 cells after high shear stress expressed significantly higher levels of Cx43 accumulation with 17% and 24% increases in fluorescent intensity compared to low and no shear conditions, respectively.

### Discussion

The success of TCR T cell therapy relies on our ability to identify TCRs that provoke strong and specific responses to tumour cells, Currently deployed methods for selecting TCR clonotypes based on the avidity of TCR and pMHC interactions are acellular and therefore incapable of probing important physiological cues for T-cell activation including a range of co-receptors and cell-cell interactions such as integrins and gap junction proteins. Here we developed a novel microfluidic device 100 that is capable of collecting TCR clonotypes based on the concept of cellular avidity, the overall binding strengths between activated T cells 50 and tumour cells 51. This technology is capable of isolating high avidity and highly cytotoxic T cells 50 in 30 minutes by simply varying applied shear stresses. Human T cells engineered with specific TCRs and primary mouse T cell variants were successfully isolated and thus, verifies that this device 100 works with physiologically relevant cells.

Previous studies revealed that the gap junction protein Cx43 accumulates at the IS in an antigen-dependent manner. Here, we found that primary mouse T cells 50 that remained adhered to melanoma cells 51 under high shear stress conditions in our device expressed higher levels of Cx43 and F-actin accumulation at the IS, which correlates to stronger IS formation and thus, stronger cellular avidities. Moreover, the device allows downstream off-device functional and genomic analyses to be conducted on collected cells. This will facilitate the sequencing of TCR chains in order to recover clones of interest for TCR T cell therapy.

Beyond the ability of this platform to isolate clonotypes based on cellular avidities, the superior optical properties of microfluidic devices and the ability to operate under physiological conditions allows the automated real-time monitoring of tens of thousands of live T cell and tumour cell interactions per device 100. As a proof of concept, in this work we validated T cell-tumour cell interactions in our device by monitoring T cell activation in real-time. T cell activation is a key determinant in initiating potent anti-tumour responses. Our data indicated that human SupT1 T cell activation increased, as avidity increased, which correlates with previous studies. This platform could be used for other applications in the future such as validation of selected TCR clonotypes or evaluation of combinatorial immunotherapy with chemotherapies, drug-loaded lipid nanoparticles, nanogels or others.

While melanoma antigens were used in this study, our platform can be easily adapted to investigate a wide range of tumours and antigens. Recently identified mutation-derived neoantigens are only found on the surface of tumour cells 51 and thus, may provide a safer route for effective T cell therapy. Current strategies to find cells with optimal avidity to neoantigens is even more laborious than self-antigens, as every patient expresses different neoantigens. This platform could therefore potentially accelerate the identification of suitable patient-specific neoantigen T cell candidates.

Finally, a shift from non-physiologically multimer/tetramer approaches to novel technologies such as microfluidics may provide faster, more accurate and physiologically relevant environments for the selection of optimal T cell clones. This provides great potential for improving current immunotherapeutic strategies as well as a step towards precision cancer medicine.

### Summary

The microfluidic devices 100 disclosed herein have the potential to improve current T cell selection strategies to rapidly identify rare high avidity T cell clones for successful adoptive immunotherapies. The device 100 developed in this work provides the ability to analyse thousands of cell-cell interactions in real-time under precisely controlled environments. This provides more intuitive analysis on physiological T cell-tumour cell interactions that is difficult to achieve with acellular conventional methods. T cells and tumour cells used in this study were obtained from pre-defined human or mouse models. However, this device has the potential to isolate rare high avidity T cell clones from a pool of heterogeneous T cells extracted from a cancer patient.

The present disclosure relates to a shear-induced microfluidic device suitable for collecting, for example, human and primary mouse T cell variants based on cellular avidity to specific tumour cells. Using this device, the highest cellular avidity and most cytotoxic primary TCR T cells can be isolated with 100% purity. The device is simple and fast to operate, with thousands of T cells processed within 30 minutes. Therefore, this strategy has the potential to fast track the therapeutic process by accelerating the isolation of optimal TCR T cell clones based on functional live-cell interactions, and may have promising applications in precision cancer medicine.

The present disclose enables antigen-presenting cells to be seeded in a microfluidic device, flowing over T cells with specific TCRs to allow them to bind, and then applying a defined and increasing shear stress across the device to detach cells with increasing avidity. Cells can be collected and the TCRs identified to select optimal TCRs, potentially in a personalised cancer medicine modality. Beneficially, the apparatus and methods of the present disclosure are capable of probing thousands-millions of interactions at once, greatly increasing the throughput compared to conventional apparatus and methods.

It will be appreciated, therefore, that the present disclosure allows the selection of optimal TCR candidates for cancer immunotherapies using human cells. This may accelerate the adoptive cell therapy drug discovery pipeline, potentially eliminate candidates destined to fail in clinical trials (resulting in significant cost savings) and enables personalised cancer medicine. The apparatus of the present disclosure is faster and more accurate than cell-free tetramer based methods, and is able to collect and process cells in far greater numbers.

### Modifications and Alternatives

Detailed embodiments and some possible alternatives have been described above. As those skilled in the art will appreciate, a number of modifications and further alternatives can be made to the above embodiments whilst still benefiting from the inventions embodied therein. It will therefore be understood that the invention is not limited to the described embodiments and encompasses modifications apparent to those skilled in the art lying within the scope of the claims appended hereto.

For example, whilst in certain embodiments above the first cells have been described as tumour cells and the second cells have been described as T cells, in alternative embodiments the first cells may be T cells and the second cells may be tumour cells. In yet further alternative embodiments, the T cells and/or tumour cells may be replaced by other suitable types of cell. For example, the T cells may be replaced by macrophages.

Whilst in certain embodiments above the channel 12 has been described as having a height of 0.1 mm (in the z direction), channel heights greater than 0.5 mm may also be used, for example up to 10 mm in height. Similarly, whilst in certain embodiments above the channel 12 has been described as having a length of 50 mm (in the x direction), it will be appreciated that the channel may be longer or shorter than 50 mm.

1. A microfluidic device for discriminating between second cells, as a function of binding strength between first cells and the second cells, the device comprising:
a microfluidic channel, a base of the channel comprising a substrate;
wherein the channel is configured to receive first cells, for adhesion of the first cells to the substrate;
receive second cells, for adhesion of the second cells to the first cells; and
receive a fluid, for fluid flow over the second cells; and wherein
a transverse cross-section of the channel is configured for application of a substantially uniform shear stress to the second cells that are adhered to the first cells in at least a central region of the channel as the fluid flows along the channel, to detach at least some of the second cells from the first cells, and to cause the detached second cells to flow towards a downstream end of the channel.

2. The microfluidic device according to claim 1, wherein a width of the channel is between 10 and 50 times greater than a height of the channel, for example 10 times, 20 times or 30 times greater than the height of the channel;
optionally wherein the channel has a generally rectangular transverse cross-section.

3. (canceled)

4. (canceled)

5. The microfluidic device according to claim 1, further comprising:
a first flow path arranged to convey fluid from the central region of the channel into a collection outlet;
a second flow path arranged to convey fluid from a first side region adjacent a first side wall of the channel into a waste outlet; and
a third flow path arranged to convey fluid from a second side region adjacent a second side wall of the channel into a waste outlet;
optionally wherein:
the first flow path is coupled to the central region of the channel at the downstream end of the channel,
the second flow path is coupled to the first side region of the channel at the downstream end of the channel, and
the third flow path is coupled to the second side region of the channel at the downstream end of the channel;
optionally wherein the second and third flow paths are connected to a common waste outlet.

6. (canceled)

7. The microfluidic device according to claim 5, wherein a fluid resistance of the first flow path relative to fluid resistances of the second and third flow paths is configured for flow of a predetermined fraction of the fluid into the waste outlet(s);
optionally wherein the second and third flow paths have greater fluid resistance than the first flow path.

8. (canceled)

9. The microfluidic device according to claim 7, wherein the dimensions of the first flow path, the dimensions of the second flow path and the dimensions of the third flow path are configured for flow of the predetermined fraction of the fluid into the first, second and third flow paths;
optionally wherein the second and third flow paths are longer and/or have smaller transverse cross-sectional areas than the first flow path.

10. (canceled)

11. (canceled)

12. The microfluidic device according to claim 5, wherein the device is configured such that fluid that flows within a predetermined distance from the side walls of the channel is diverted into the waste outlet(s);
optionally wherein the predetermined distance is 10% of the width of the channel.

13. (canceled)

14. The microfluidic device according to claim 12, wherein the predetermined distance is 0.1 mm.

15. The microfluidic device according to claim 1, wherein
the first cells are T cells, for example human T cells, and
the second cells are tumour cells, for example human tumor cells; or wherein:
the first cells are tumour cells, for example human tumor cells, and
the second cells are T cells, for example human T cells.

16. (canceled)

17. The microfluidic device according to claim 1, wherein the height of the channel is less than 0.5 mm, for example 0.1 mm;
or wherein the substrate is modified with a plasma treatment or other non-protein based treatment;
or wherein the substrate is a plastic substrate.

18. (canceled)

19. (canceled)

20. The microfluidic device according to claim 1, wherein the substrate is a glass substrate;
optionally wherein the glass substrate is coated with poly-L-lysine or fibronectin.

21. (canceled)

22. The microfluidic device according to claim 1, wherein the fluid comprises at least one of a biological fluid, balanced salt solution, basal media, complex media, a mineral or other cell-compatible oil;
or wherein the fluid comprises a shear thinning fluid (for example, blood) or a shear thickening fluid;
or wherein the first cells form a monolayer on the substrate;
or wherein the length of the channel is greater than 10 mm, for example 50 mm.

23. (canceled)

24. (canceled)

25. (canceled)

26. Apparatus comprising the microfluidic device according to claim 1.

27. The apparatus according to claim 26, further comprising a controller for controlling a flow rate of the fluid into the channel to control the substantially uniform shear stress;
the apparatus optionally further comprising a syringe pump for controlling the flow rate of the fluid into the channel to control the substantially uniform shear stress;
optionally wherein the controller is configured to sequentially change the flow rate of the fluid into the channel between a plurality of predetermined flow rates.

28. (canceled)

29. The apparatus according to claim 26, further comprising a sorting stage towards the downstream end of the channel for sorting groups of the second cells based on the applied shear stress at which the second cells detached from the first cells;
the apparatus optionally further comprising a downstream analysis stage for analysing the sorted groups of detached second cells.

30. (canceled)

31. (canceled)

32. The apparatus according to claim 29, wherein the controller is configured to sequentially change the flow rate of the fluid into the channel between a plurality of predetermined flow rates, and wherein the sorting stage is configured to sort the second cells into groups of the second cells that are detached from the first cells at each of the corresponding predetermined flow rates.

33. The apparatus according to claim 26, further comprising an imaging device configured for imaging the first and/or second cells in the channel.

34. A method for discriminating between second cells, as a function of binding strength between first cells and the second cells, the method comprising, in a microfluidic channel, a base of the channel comprising a substrate:
receiving first cells, for adhesion of the first cells to the substrate,
receiving second cells, for adhesion of the second cells to the first cells; and
receiving a fluid, for fluid flow over the cells; wherein
a transverse cross-section of the channel is configured for application of a substantially uniform shear stress to the second cells that are adhered to the first cells in at least a central region of the channel as the fluid flows along the channel, thereby detaching at least some of the second cells from the first cells, and causing the detached second cells to flow towards a downstream end of the channel.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. The method according to claim 34, the method further comprising:
controlling a flow rate of the fluid into the channel to control the substantially uniform shear stress;
sorting groups of the second cells based on the applied shear stress at which the second cells detached from the first cells; and
analysing the sorted groups of detached second cells;
wherein the analysing includes determining an avidity between at least one of the second cells and the first cells based on the applied shear stress at which the at least one second cell detached.

48. (canceled)

49. (canceled)

50. (canceled)

51. The method according to claim 34, further comprising imaging the second cells that detach from the first cells.

52. The method according to claim 34, wherein the second cells are allowed to adhere to first cells for a period of between 5 and 10 minutes, for example 10 minutes, under static conditions.
